(19) United States
(12) Patent Application Publication
(10) Pub. No.: US 2009/0232916 A1
(43) Pub. Date: Sep. 17, 2009

(54) FOOD PRODUCTS FOR DIABETICS

(76) Inventors: Avidor Shulman, Kiryat Tivon (IL); Dori Pelled, Hod Hasharon (IL); Tzafra Cohen, Haifa (IL)

Correspondence Address: Fleit Gibbons Gutman Bongini & Bianco PL, 21355 EAST DIXIE HIGHWAY, SUITE 115, MIAMI, FL, 33180, US

(21) Appl. No.: 11/573,457

(22) PCT Filed: Aug. 9, 2005
(86) PCT No.: PCT/IL05/00854

§ 371 Date: Jun. 6, 2007

(30) Foreign Application Priority Data
Aug. 9, 2004 (IL) 163418

(51) Int. Cl. A61K 36/16; A61K 31/20; A61K 31/35; A61K 31/397; A61K 31/66

(52) U.S. Cl. 424/752; 514/558; 514/460; 514/210.02; 514/121

(57) ABSTRACT

Disclosed is a novel food product characterized by a low glucose or glucose free content, a balanced functional fat content, and a proactive agent aimed for the diabetic and diabetic-prone populations. The food product of the invention is a functional food which may be used clinically to lower the lipid level in people suffering from an imbalanced lipid profile and which may progress towards diabetes complications and coronary vascular disorders. In particular embodiments the proactive agent can be any of a naturally occurring lipid, a synthetic or mimetic lipid which is not digestible by humans and interferes with body weight gain/loss, plant extracts and substances derived therefrom, antioxidants, animal-derived substances, minerals and pharmaceuticals, and any mixture thereof.

### FIELD OF THE INVENTION

The present invention relates to the field of functional foods and clinical foods. More specifically, the present invention provides novel food products with a balanced and functional fat content, aimed for the diabetic and diabetic-prone populations.

### BACKGROUND OF THE INVENTION

All publications mentioned throughout this application are fully incorporated herein by reference, including all references cited therein.

Diabetes mellitus Type 2, is a multiple aetiology metabolic disorder, characterized by chronic hyperglycemia with disturbances of carbohydrate, fat and protein metabolism, resulting from a partial or absolute deficiency of insulin. A deficiency of insulin in the body results in diabetes mellitus.

Diabetes is a common disease and according to the Center for Disease Control and Prevention (CDC) survey in 2002, 6.3% of the American population suffers from diabetes (21.1% out of the adult population). About 1.3 million new cases (aged above 20 years) are reported each year and it is estimated that only about half the people who currently have diabetes are diagnosed.

The effects of diabetes mellitus include long-term damage, dysfunction and failure of various organs. Often, diabetes symptoms are not as severe; however long-term hyperglycemia results in cardiovascular disease (CVD, see below), retinopathy (blindness), nephropathy (renal failure) and/or neuropathy (foot ulcers, amputation etc.). Accelerated decline of cognitive function and other brain functions or tissues damage is often induced by or accompanied to diabetes mellitus.

The metabolic abnormalities in Type 2 diabetes mellitus, which result from primarily insulin resistance in liver, muscle, and adipose tissues, lead to an increase in the blood sugar and hyperinsulinemia (high insulin blood level). Insulin resistance also leads to a disturbance of virtually all the regular cardiovascular-associated risk factors, blood pressure and lipoproteins in particular, which will increase atherosclerosis (a process based on fat metabolism abnormality) incidence. The high insulin levels seen in insulin resistance may also directly promote the development of atherosclerosis.

Accumulation of fat in the abdominal area, particularly in the visceral fat compartment, is associated with the development of insulin resistance and increased risk to display complications such as diabetes mellitus. The relationship between fatty acid content of triglyceride (TAG), visceral adipose tissue accumulation, and metabolic components of the insulin resistance syndrome have been extensively investigated [Tremblay, A. J. et al. (2004) Metabolism 53, 310-317].

The adipose tissue is not simply an energy storage organ but also a secretory organ. The regulatory substances produced by adipocytes, which include leptin, resistin, and adiponectin, may contribute to the development of insulin resistance. Furthermore, the elevated level of free fatty acids occurring in obesity, had been implicated in the development of insulin resistance [Grundy S M. (2004) J Clin Endocrinol Metab, 89(6):2595-2600].

Diabetes patients are three to five times more likely to have coronary heart disease (CHD) and stroke than non-diabetic patients. In fact, heart disease is the leading cause of diabetes-related deaths.

Numerous studies have shown that oxidative stress may also play a major pathophysiological link between CVD and Type 2 diabetes [Baynes J. W. and Thorpe S. R. (1999) Diabetes 48:1-9]. Oxidative stress, a relative increase in free radicals, is a direct consequence of hyperglycemia. Reactive oxygen molecules created by this metabolic imbalance activate monocytes and macrophages triggering the proliferation of vascular smooth muscle cells and an overall diabetic angiopathy. One of the main outcomes of this process is the generation of high levels of oxidized-LDL species, which are taken in by macrophages through scavenger receptors (and not native LDL receptors), to give rise to foam cells, the hallmark of early atherosclerosis [Griendling, K. K. and FitzGerald, G. A. (2003) Circulation 108(17): 2034-40].

The dyslipidemia associated with insulin resistance and Type 2 diabetes is characterized by elevated triglycerides, low levels of HDL cholesterol, an increase in the proportion of small, dense, and potentially more atherogenic LDL cholesterol particles. These abnormalities are present for years before diabetes mellitus is diagnosed clinically. Elevated triglyceride levels, reduced HDL cholesterol, and increased atherogenic LDL cholesterol levels are all risk factors for CVD.

Several population based studies have shown that Type 2 diabetes also increases the risk of dementia. Cognitive decline is an intermediate stage between normal ageing and dementia. As dementia may be most effectively delayed in its initial stages, identifying diabetes as a modifiable risk factor for early cognitive decline could be of major importance. Recently, a large study focused on elderly women with Type 2 diabetes described increased odds of poor cognitive function and substantial cognitive decline in these patients, compared with women without diabetes [Logroscino et al, (2004) BMJ. 328:548-553].

Fatty acid differences, including docosahexaenoic acid (DHA) have been shown in the brains of Alzheimer's disease (AD) patients as compared with normal age-matched individuals. Furthermore, low serum DHA is a significant risk factor for the development of AD. It was recently argued by Muskiet and colleagues [Muskiet F. A. et al. (2004) J. Nutr. 134:183-6] that DHA is in fact an essential fatty acid, also in light of its relation to cognitive functions. Indeed, other investigators stated that dietary intake of omega-3 fatty acids and weekly consumption of fish may reduce the risk of incident Alzheimer's disease.

Diabetes impairs essential fatty acid metabolism by decreasing activities of Δ6 and Δ5 desaturases, enzymes that convert dietary linoleic acid and alpha-linolenic acid to long-chain polyunsaturated fatty acids (PUFA), including gamma-linolenic acid, arachidonic acid (AA), eicosapentaenoic acid (EPA), and DHA. As a result, AA and DHA levels are reduced in membrane phospholipids of several tissues, including erythrocyte and sciatic nerve. It was demonstrated recently that dietary supplementation with fish oil, containing EPA and DHA, partially prevented the diabetes-induced decrease in nerve conduction velocity, a physiological marker of diabetic neuropathy. This was further correlated in a recent report that presented evidence for a marked neuro-protective effect of DHA on diabetic neuropathy. Furthermore, there are several reports describing the beneficial effects of fish oil-derived omega-3 fatty acids supplementation on serum triglycerides, HDL, lipid peroxidation and antioxidant enzymes, which may lead to decreased rate of occurrence of vascular complications and minimize the cognitive decline in diabetes.

The importance of prevention of diabetes in high-risk individuals is highlighted by the substantial and worldwide increase in the prevalence of diabetes in recent years. Genetic susceptibility appears to play a powerful role in the occurrence of Type 2 diabetes in certain populations. However, given that population gene pools shift quite slowly, the current epidemic likely reflects marked changes in lifestyle that are characterized by decreased physical activity and increased energy consumption. Weight reduction, exercise, and dietary modifications often correct the hyperglycemia of Type 2 diabetes. Therefore, changes in the population habits, including nutrition habits, may decrease the risk of developing diabetes and diabetes complications.

Diabetes patients are first of all treated, mostly through medication, in regard to their hyperglycemia and glucose control. This is aimed to prevent long-term complications. As mentioned above, the long-standing elevation of blood glucose causes chronic complications of diabetes-premature atherosclerosis, retinopathy, nephropathy, and neuropathy.

In some cases their cardiovascular health is addressed, again mainly through medications, in regard to their hyperlipidemia, mainly hypercholesterolemia. Little or no treatment is provided for their hypertriglyceridemia. An important risk factor, associated and even induced with their metabolic disorder is blood oxidative stress. This risk factor leads to atherosclerosis, and together with hyperlipidemia, it is one of the main causes of mortality of cardiovascular disorders among diabetes patients.

Diabetic patients are recommended to modify nutrient intake and lifestyle as appropriate for the prevention and treatment of obesity, dyslipidemia, cardiovascular disease, hypertension, and nephropathy. The American Diabetes Association has recently published goals of medical nutrition therapy aimed to attain optimal metabolic outcomes including blood glucose, lipid profile and blood pressure in order to prevent and treat the chronic complications of diabetes [Franz M J, Et al, Diabetes Care 25:148-198, 2002; American Diabetes Association Position Statements “Nutrition Principles and Recommendations in Diabetes” (2004]. Nevertheless, diabetics are typically treated only in regard to glucose control and their hyperglycemia through strict diets. These diets are mainly designed to lower the amount of dietary glucose to a minimum. In most cases, these diets are not designed to promote heart health, let alone fight the main cardiovascular risk factors.

Diabetes patients are offered unique food products, especially designed to accommodate their unique dietary requirements in regard to minimal glucose levels. However, in most cases, these food products are rich in lipids, and especially triglycerides, in the form of oils and fats. These fats are added in order to compensate the lack of glucose and the poor sensorial properties of the resulting foods. Although this addition indeed yields sensorial desirable food articles, they have severe adverse effects on the diabetes patients who should fear fat-rich products almost as much as glucose-rich products.

Furthermore, due to food technology reasons, many of the fats and oils used in food products for diabetics are actually saturated fats which enhance even more the risk of the acute cardiovascular disorders of diabetics. Moreover, in many cases, food products for diabetics consist of higher levels of fats than their counterpart, “regular” food products. This difference in fat levels can reach up to 60% additional fats in diabetics' specialty foods.

Therefore, there is a great need in the market for specialized food aimed at the diabetic population, in which all its content, and not only the glucose component, will be beneficial for said population. It is important to provide diabetics, or individuals prone to diabetes, with foods wherein the fat content is appropriate for their needs. Said specialized food products shall be fat balanced, and its fat content shall even be beneficial to diabetics or people prone to diabetes.

Furthermore, it will be beneficial if diabetics will be able to consume through their specialized daily nutrition additional ingredients that may have a protecting health effect and even a pro-active beneficial health effect, especially on their cardiovascular condition and/or on different risk factors leading to the development or progress of CVD.

Thus, it is an object of the present invention to provide a food product or article with low glucose levels or glucose free and a balanced lipid profile, this food also comprising at least one diabetes-proactive dietary or pharmaceutical substance, it does not contain significantly high levels of fats and oils and, most importantly, comprises minimal amounts of saturated fats, substantially lower than the amounts currently used in diabetics foods available on the market. Other uses and objects of the invention will become clear as the description proceeds.

Despite the understanding of nutrition and medical experts of the specific health needs of diabetics, especially their cardiovascular needs, and despite the progress in food technology that may enable the use of healthier oils and fats and at smaller amounts, no one has yet combined the three (nutrition, medicine, food technology) to yield foods such as presented by the inventors. Furthermore, no diabetic food product has introduced a pro-active approach to address such cardiovascular needs.

Although the cardiovascular health needs of diabetics are perhaps the most extreme, other populations can consume and benefit from the food products of the invention. Such populations also include pre-diabetics which are prone or are at high risk of developing Type 2 diabetes and cardiovascular diseases. Pre-diabetics may be individuals with high risk to develop Type 2 diabetes in light of different conditions including genetic background, over-weight, obesity and especially abdominal obesity, specific ethnicity, previously identified as impaired fasting glucose (IFG) or impaired glucose tolerance (IGT), hypertension, dyslipidemia, history of gestational diabetes, as well as elderly individuals. Several of the risk factors may exist simultaneously, dramatically elevating the risk to develop a diabetic condition.

Other populations that may benefit from the food products of the invention are individuals diagnosed with the metabolic syndrome or Syndrome X, which is characterized by glucose intolerance, hyperinsulinaemia, dyslipidemia, hypertension, visceral obesity, hypercoagulability, and proinflammatory state. All the above mentioned symptoms define a cluster of CVD risk factors.

Similarly, CVD patients, including patients who already suffered a cardiovascular event, as well as individuals which are at high risk or prone to develop CVD due to metabolic syndrome, obesity, over weight, hypertension, dyslipidemia, etc., may benefit. Other populations with different metabolic disorders may also benefit from the food products of the invention since most metabolic disorders may lead to cardiovascular health problems and the subsequent adverse effects and symptoms. Health conscious populations who wish to consume balanced diets, especially from a fat content point of view, also having pro-active effects in the prevention or inhibition of CVD and metabolic disorders, especially diabetes or diabetes-prone subjects may also benefit from the food products of the invention.

Recent studies have highlighted the potential for intervention in IGT subjects to reduce progression to Type 2 diabetes. For example, the Diabetes Prevention Program in the United States [The Diabetes Prevention Program (1999) Diabetes Care 22:623-634] had proved that lifestyle intervention (targeting diet and exercise) over a period of three years, reduced the risk of progressing from IGT to diabetes by 58%, whereas the oral hypoglycemic drug, metformin, reduced the risk by only 31% [Larkin, M. (2001) Lancet. 358(9281):565]. Another large-scale study conducted by a Finnish diabetes prevention organization [Sarkkinen E. et al., (1996) Eur J Clin Nutr 50(9):592-8] proved that non-pharmacological lifestyle intervention at people with high risk to develop type 2 diabetes prevents or at least postpones the onset of the disease.

In other studies, it was shown that the approach aimed at high-risk individuals (for example, those with impaired glucose tolerance (IGT)) may not be sufficient in preventing all the cases of Type 2 diabetes. Data from the UKPDS indicate that pancreatic β-cell function is already substantially reduced at the time of clinical diagnosis of Type 2 diabetes [UK Prospective Diabetes Study (UKPDS) Group (1998) Lancet 352(9131):837-853; U.K. Prospective Diabetes Study Group (1995) Diabetes 44(11):1249-1258]. Consequently, therapeutic interventions with hypoglycemic drugs or with functional/medical nutrition products should be considered for prevention as well.

Current special foods for diabetics are also being consumed by populations merely seeking weight loss/control, not necessarily required from a medical or health risk point of view. These populations mistakenly interpret the no glucose/sugar labels of such products as being weight loss/control foods in the sense of low fat and/or low calorie. As mentioned above, most if not all currently available food products designed and/or recommended for the diabetic population actually have higher levels of fat, and even saturated fats, as well as high caloric content. In many cases, such a specialized food product has a higher fat content and/or caloric content than the corresponding “regular” food product (see examples). Therefore, populations seeking weight loss/control foods, as described above, can also benefit from the food products of the invention which have a balanced and even low fat content, lower levels of saturated and other un-healthy fats, as well as optionally including ingredients which can improve health, as described in the invention.

Surprisingly, the food products of the invention may also have a preventive effect in regard to the development of Type 2 diabetes. Consumption of the food products, having said balanced fat content, low levels of glucose, and optionally at least one active ingredient can prevent or inhibit the onset of diabetes. A diet based on the food products of the invention or rich with such food products can control glucose metabolism and even insulin secretion. Consumption of such a diet can decrease the propagation of pancreatic β cells deterioration process and might even lead to a reduction or reversion of insulin resistance. Diets based on the food products of the invention may stabilize the glycemic state, control the insulin secretion, and furthermore reduce the accumulation of visceral fat. Abdominal fat accumulation is considered to be the underlying process accompanies the progression of Type 2 diabetes; therefore reduction of fat accumulation in the abdomen may prevent or postpone the diabetes onset and progression.

### SUMMARY OF THE INVENTION

In a first aspect, the present invention relates to a food product or article characterized by having a low glucose content or being glucose free, a balanced fat content and comprising at least one dietary or pharmaceutical substance which is proactive towards diabetes and any of its complications, and/or towards a condition leading to diabetes, for addressing health needs of diabetics or for preventing onset of diabetes in healthy individuals or individuals prone to diabetes.

In said food product the diabetes-proactive dietary or pharmaceutical substance may be any one of a naturally occurring lipid, a synthetic or mimetic lipid which is not digestible by humans and inhibits body weight gain, plant extracts and substances derived therefrom, antioxidants, animal-derived substances, minerals and pharmaceuticals, and any mixture thereof, said substances being optionally dispersed or dissolved in an edible oil or fat.

In a particular embodiment, the lipid in said food product may be any one of diacylglycerols, particularly 1,3-diacylglycerols, phytosterols and phytosterol esters, phytostanols and phytostanol esters, polycosanols, omega-3 fatty acids and their derivatives, particularly long-chain polyunsaturated fatty acids (LC-PUFA), polyunsaturated fatty acids (PUFA), particularly alpha-linolenic acid, and conjugated linolenic acid (CLA); and/or a non-digestible synthetic lipid or lipid mimetic is an alpha branched triglyceride or olestra, respectively.

The plant extract or substance derived therefrom component in said food product may be any one of garlic extract, soy protein, soy isoflavone, lycopene, lutein, zeaxanthin, vitamin C, vitamin E and other tocopherols, beta-carotene, polyphenols, particularly hydroxytyrosol, folic acid, vitamin B6 and vitamin B12; the antioxidant is any one of rosemary extract, lycopene, zeaxanthin, selenium, zinc, vitamin C, vitamin E and other tocopherols, coenzyme Q₁₀, beta-carotene, polyphenols, particularly hydroxytyrosol; the animal-derived substance may be whey protein or a casein; the mineral is calcium, selenium or zinc; and said pharmaceutical component may be any one of statins, ezetimibe, a drug controlling lipids profile or other biomarker related to cardiovascular diseases.

The food product in the invention may comprise at least one diabetes-proactive substance, although it may also include any mixtures thereof, optionally dispersed or dissolved in an edible oil or fat.

In a more particular embodiment, said diabetes-proactive dietary substance is a mixture of phytosterol and/or phytostanol ester(s) with 1,3-diacylglyceride(s), optionally dispersed or dissolved in an edible oil or fat.

In a special embodiment, said food product may further comprise at least one pharmaceutical drug, particularly a drug which is proactive towards diabetes, any condition leading to diabetes or any diabetic complication.

In one embodiment, the food article of the invention is functional in the treatment and/or prevention of cardiovascular disease (CVD) and/or its risk factors hyperlipidemia, dyslipidemia, oxidative stress and atherosclerosis, particularly in diabetic or diabetes-prone individuals.

In another embodiment said food product is functional in the treatment and/or prevention of hyperlipidemia, dyslipidemia, oxidative stress and atherosclerosis, particularly in diabetic or diabetes-prone individuals.

In a particular embodiment, the food product of the invention is functional in at least one of the following: reducing the total cholesterol serum level, reducing the non-HDL cholesterol serum level, reducing the total cholesterol/HDL ratio and reducing triglycerides serum level in an overweight, and/or obese, and/or diabetic, and/or diabetic-prone subject.

In another particular embodiment, said food product is functional in reducing the body weight, and/or inhibiting body weight gain, and/or reducing insulin resistance in an obese and/or in a subject with metabolic imbalances such as diabetes and/or an individual prone to diabetes or obesity.

In a special embodiment, said food product is also functional in remodeling body fat distribution, suppressing white adipose tissue (WAT) accumulation and reducing visceral fat accumulation in an obese and/or overweight subject and/or diabetic subject and/or pre-diabetic subject and/or an individual prone to diabetes or obesity.

In a broad aspect, the food product of the invention is functional in reducing the risk of life threatening long term diabetes complications and deterioration of quality of life in an obese and/or diabetic subject. In a particular case, said complications are from macrovascular, microvascular or neurological origin and it is selected from the group of retinopathy, nephropathy, diseases of the large vessels supplying the legs (lower extremity arterial disease), coronary heart or artery diseases, cerebrovascular diseases and disturbed neural function afflictions, decline of cognitive functions, or any other condition which may lead to blindness, end-stage kidney disease (ESRD) and amputations, myocardial infractions and stroke.

In another aspect, the food product of the invention is functional in ameliorating hyperinsulinemia in an insulin-resistant subject. In a particular case, said food product is functional in ameliorating hyperinsulinemia or preventing progression of insulin resistance in an obese subject prone to develop diabetes.

In a further embodiment, the food product of the invention further comprises active agents which are functional in the prevention and/or treatment of acute cognitive decline and said active agent is any of phosphatidylserine, ginko biloba, brahmi (Bacopa monnieri) or omega-3 containing fats.

In an additional embodiment, the food product of the invention further comprises active agents which are functional in the prevention and/or treatment of acute ophthalmic conditions associated with Type 2 diabetes, wherein said active agents are vitamins, antioxidants, and other natural eye-health promoting ingredients or extracts.

Finally, the food product described herein may be one of dairy products, bakery products, condiments, beverages and drinks, snacks, candies, ice-creams and frozen desserts, morning cereals, nutrition bars, snack bars chocolate products, prepared foods, grain products and pasta, soups, sauces and dressings, confectionery products, oils and fats products, dairy and milk drinks, soy milk and soy dairy-like products, frozen food products, prepared meals and meal replacements, meat products, cheeses, yoghurts, breads and rolls, yeast products, cakes and cookies and crackers.

### BRIEF DESCRIPTION OF THE FIGURES

FIG. 1: Plasma total cholesterol levels in diabetic Psammomys obesus fed with mono and polyunsaturated fatty acid nutritional diets

Abbreviations: HSFA—10:1 Highly enriched with saturated fatty acids; HMUFA—oil highly enriched with monounaturated fatty acids; T. Chol—total cholesterol; mg/dL—milligram per deciliter.

FIG. 2: Plasma non-HDL cholesterol levels in diabetic Psammomys obesus fed with mono and polyunsaturated fatty acid nutritional diets

Abbreviations: HSFA—10:1 Highly enriched with saturated fatty acids; HMUFA—oil highly enriched with monounaturated fatty acids; Non-HDL. Chol.—non-high density lipoprotein cholesterol; mg/dL—milligram per deciliter.

FIG. 3: Plasma total cholesterol/HDL ratio in diabetic Psammomys obesus fed with mono and polyunsaturated fatty acid nutritional diets

Abbreviations: HSFA—10:1 Highly enriched with saturated fatty acids; HMUFA—oil highly enriched with monounaturated fatty acids; T. Chol.—total cholesterol; HDL—high density lipoprotein cholesterol; Rat.—ratio; mg/dL—milligram per deciliter.

FIG. 4: Plasma total cholesterol levels in “pre-diabetic” Psammomys obesus fed with monounsaturated fatty acids nutritional diet

Abbreviations: H.E.—High energy; T. Chol.—total cholesterol; mg/dL—milligram per deciliter.

FIG. 5: Plasma non-HDL cholesterol levels in “pre-diabetic” Psammomys obesus fed with monounsaturated fatty acids nutritional diet

Abbreviations: H.E.—High energy; Non-HDL. Chol.—non-high density lipoprotein cholesterol; mg/dL—milligram per deciliter.

FIG. 6: Plasma total cholesterob=L ratio in “pre-diabetic” Psammomys obesus fed with monounsaturated fatty acids nutritional diet

Abbreviations: H.E.—High energy; T. Chol.—total cholesterol; HDL—high density lipoprotein cholesterol; Rat.—ratio; mg/dL—milligram per deciliter.

FIG. 7: Body weight of “pre-diabetic” Psammomys obesus fed with monounsaturated fatty acids nutritional diet

Abbreviations: H.E.—High energy; E.P.B.W.—endpoint body weight; g—grams.

FIG. 8: Epididymal fat weight of “pre-diabetic” Psammomys obesus fed with monounsaturated fatty acids nutritional diet

Abbreviations: H.E.—High energy; E.P.E.F.—endpoint epididymal fat weight; g.—grams.

FIG. 9: Epididymal fatliver weight ratio of “pre-diabetic” Psammomys obesus fed with monounsaturated fatty acids nutritional diet

Abbreviations: H.E.—High energy; E.F.W.—epididymal fat weight; Liv. W.—liver weight; Rat.—ratio; %—percentage.

FIG. 10: Plasma insulin levels in “pre-diabetic” Psammomys obesus fed with monounsaturated fatty acids nutritional diet

Abbreviations: H.E.—High energy; Ins.—insulin; micU/ml—micro unit per millfiter.

FIG. 11: Plasma insulin levels relative to body weight in “pre-diabetic” Psammomys obesus fed with monounsaturated fatty acids nutritional diet

Abbreviations: H.E—High energy; Ins.—insulin; B.W.—body weight; g—grams; micU/ml—micro unit per milliliter.

FIG. 12: Plasma insulin levels relative to plasma glucose levels in “pre-diabetic” Psammomys obesus fed with monounsaturated fatty acids nutritional diet

Abbreviations: H.E.—High energy; Ins.—insulin; Glu.—glucose; mg/dL—milligram per deciliter; micU/ml—micro unit per milliliter.

### DETAILED DESCRIPTION OF THE INVENTION

In view of the pressing demand of the diabetic and diabetic-prone populations for specialized food articles which are truly appropriate for their needs, as well as for health solutions addressing their unattended health risk factors, the present inventors have developed a novel food product platform.

Thus, in a first aspect, the present invention relates to a food product or article which has a low glucose content or is preferably glucose free, characterized in that its fat content is balanced, based on small amounts of non saturated oils and fats, and that it comprises at least one diabetes-proactive dietary or pharmaceutical substance for addressing health needs of diabetics, including conditions leading to diabetes and diabetic complications, or for preventing onset of diabetes in healthy individuals or individuals prone to diabetes.

A “diabetes-proactive dietary substance” as used herein, is any nutritional or dietary substance which can be consumed on a daily basis, provided as a day-to-day food supply, without having any adverse effect that may compromise the health condition of a diabetic individual, aggravate or induce any risk condition related to diabetes, and does not promote the onset of diabetes in diabetic prone individuals, and this term also encompasses pharmaceuticals and drugs.

The “diabetes-proactive dietary product” is characterized by having low or no glucose content, a balanced fat content, preferably low levels of saturated fats or any other harmful fats, such as trans fats, together with the proactive ingredient.

The proactive health benefit is achieved by being capable of improving or preventing dyslipidemia, hypertriglyceridemia, hypercholesterolemia, oxidative stress, high insulin blood levels, abdominal obesity, or other risk factors related to CVD, usually present in diabetic and diabetes-prone individuals.

In a preferred embodiment the diabetes-proactive dietary substance is a mixture of phytosterol and/or phytostanol ester(s) (PS-E) with 1,3-diglyceride(s) (DAG), optionally dispersed or dissolved in an edible oil or fat. Some favored mixtures may combine PS-E(s) and DAG(s), preferably with a higher phytosterols esters content relatively to DAG.

Furthermore, said food product may contain pharmaceutical active ingredients for addressing health needs of diabetics or for preventing onset of diabetes in healthy individuals or individuals prone to diabetes. In a preferred embodiment said pharmaceutical active ingredient aims to lower cardiovascular risk factors, such as dyslipidemia and/or abnormal lipid profile (for example high cholesterol blood level), frequent in diabetics or diabetic-prone individuals.

Some of the preferred pharmaceutical active ingredients may include, but are not restricted to, statins, bile acid sequestrants and ezetimibe, which are effective in lowering LDL amounts by inhibiting cholesterol biosynthesis, preventing re-absorption of bile acids or by preventing absorption of dietary and biliary cholesterol together. Any other drug capable of controlling the lipids profile or other biomarker associated with cardiovascular diseases may be considered.

The food product of the invention contains at least one dietary active ingredient and/or at least one pharmaceutically active ingredient which may confer an additive health benefit or alternatively a synergistic health benefit.

Basically, the market and producers of special foods for diabetics concentrate on food products which are traditionally rich in sugar or sweet foods, such as candies, chocolate, cookies, fruit jams, cakes, etc. In these products the emphasis is on taking out the sugar without taking out the sensorial quality the consumer expects from such indulgence foods. This again emphasizes how the impact of nutrition on the diabetics' health needs, especially those needs which are not directly related to their insulin resistance is basically overlooked and/or ignored. Thus, it is the purpose of this invention to relate to the full scope of food products consumed by individuals and making them more appropriate to consumption by diabetics or diabetes-prone individuals. In a specific embodiment the invention relates to food products which are traditionally produced with high levels of fat, and especially saturated fat, and do not contain any proactive ingredients to address the CVD risk factors of diabetics, some of which are thriving because of poor nutrition. It should be emphasized that many food products nowadays are indeed designed with low fat content and even with low caloric values with the aid of non-sugar carbohydrates, artificial sweeteners, and other food additives, in response to the demand for food products that would not contribute to weight gain and obesity. Nevertheless, these usually have a high percentage of saturated fat from the total fat content of the food product and additionally they do not proactively contribute to the various health risk factors related to diabetics or which are more pronounced in diabetes patients or which are more crucial to pre-diabetics.

By low glucose content as used herein is meant a glucose content of between 0 up to 5% glucose, preferably from 0 up to 0.5%, most preferably below 0.1%.

By balanced fat content as used herein is meant is meant a fat or oil content of less than about 10%. Generally, the fat content of the food product will be similar or preferably lower than the fat content found in the corresponding food product, which is not special for the diabetic or pre-diabetic population. Furthermore, the fat content should be comprised of little or no saturated fats, preferably less than about 25% of the total fat, more preferably below 10%. Moreover, the fat component of the diet may particularly comprise monounsaturated or polyunsaturated fatty acids, preferably more than about 20%, more preferably above 30%, even more preferably, above 40% of the total fatty acid moieties.

Most importantly, the lipid profiles of the food article of the invention consist of minimal amounts of saturated fats, and certainly considerably lower than the amounts currently used in food articles for diabetics, which are, for example, up to 50, and even up to 75% of the total fat. This can be achieved through modern food technology methodologies and additives that enable to reduce the levels of saturated fats, usually needed from technical or texture reasons.

The food products of the invention may include a variety of dietary active ingredients. Preferably, said ingredients contribute health benefits related to cardiovascular health or addressing risk factors of CVD in general, are also considered functionally active in the treatment and/or prevention of CVD in diabetic or diabetic-prone individuals.

Phytosterol and/or phytostanols are phyto-derivatives of cholesterol clinically demonstrated to reduce blood cholesterol levels. Although they are structurally similar to cholesterol, they are not synthesized by the human body; they are very poorly absorbed by the human intestine and tend to decrease the absorption of both dietary and endogenously derived cholesterol in the intestine.

Phytosterols and/or phytostanols also include a variety of derivatives, such as fatty acid esters, as well as other ester derivatives. These ingredients have been shown to confer their health benefits at different daily intake levels. Current recommendations regarding the supplementation of phytosterols or their derivatives state an intake of 0.8 g of phytosterols, and higher levels of the stanol derivatives. Intake levels of different sterol derivatives are calculated accordingly. Phytosterols and/or phytostanols are generally produced from soybean or wood and other sources are available [Law M. (2000) BMJ 320; 861-4].

Diglycerides (DAG) are mono-hydrolyzed derivatives of triglycerides, also known as oils and fats. DAG, mainly 1,3-diglycerides, as compared to TAG, have been previously shown to lower fasting and postprandial serum TAG concentrations in humans and in animal model. When consumed in large amounts, DAG lowers body weight, total fat and abdominal fat stores. Therefore consumption of DAG should be considered for treating obesity [Tada, N. (2004) Curr Opin Clin Nutr Metab Care 7, 145-149].

DAGs have been marketed as food products only as cooking oil and certainly were not used in the nutrition of diabetics. DAG have been shown to confer their health benefits mainly in cases where they were used to replace most or all of the daily dietary fat intake of individuals. In most cases, levels above 10 g/day and even higher than 40 g/day were used. Much lower levels of DAG have been shown to confer different CVD related benefits in specific combinations with fatty acid esters of phytosterols (see below). The food products of the invention may indeed utilize DAGs to replace part of their fat content and even up to replacing the whole fat fraction.

Specific phytosterol esters and DAGs combinations optionally dissolved or dispersed in oils and/or fats, and their attributes have been described in detail in co-pending co-owned WO 01/75083 and WO 03/06444. In brief, these formulations comprise a combination of DAG, mainly 1,3-DAG(s) and phytosterol and/or phytostanol ester(s) (PSE) dissolved or dispersed in an edible oil and/or fat. Preferably said oil is olive oil, canola oil or fish oil. This composition has been shown to reduce blood cholesterol and triglycerides levels and assist in preventing or treating blood oxidative stress, thus inhibiting the atherosclerosis cascade. Furthermore, this composition has been shown to maintain and preserve the body's natural defense mechanisms, such as the paraoxonase 1 (PON1) enzyme, whose activity is dramatically reduced when oils and fats are consumed in the diet. Moreover, equal amounts of these phytosterol-esters and DAG in fats and oils maintained normal activity levels of PON1 in ApoE° knockout mice (WO 2004/069150). These combinations of phytosterol esters and DAG can be easily used to replace part, most, or all of the fat content of the food products of the invention. The level of the phytosterol esters and DAG combination in each food item of the invention can be controlled to provide phytosterol esters levels according to current recommended daily allowances (RDA) in such a way that, at least, the full RDA can be achieved through the consumption of several servings of a specific food product, through the consumption of a single serving of different food products all containing the phytosterol esters/DAG combinations or in a single serving of a specific food product.

It has now been surprising found that these combinations are also beneficiary for diabetics or pre-diabetics, as will be shown in the examples below.

Omega-3 fatty acids, such as alpha-linolenic (18:3) acid and especially docosahexaenoic acid (22:6, DHA) and eicosapentaenoic acid (20:5, EPA) have been shown to have beneficial effects on cardiovascular health. These fatty acids, produced from different marine animals and organisms, particularly from cold water fish, as well as from different microbial and algal sources, have been shown to lower blood triglycerides levels and render anti-inflammatory, antithrombotic and immunomodulatory effects.

Omega-3 linolenic acid, such as found in flax seed oil, has also been shown to have beneficial effects on lowering the risk of MI and fatal ischemic heart disease in women and in men. Long chain omega-3 fatty acids (DHA, EPA) have been shown to exert beneficial effects and current recommended intakes are above 650 mg/day and even higher.

The omega-3 fatty acids and especially their long-chain polyunsaturated members (DHA and EPA) suffer from stability problems in light of their oxidation sensitivity. The same oxidation problem is also related to organoleptic problems in which products containing said fatty acid suffer from “fish-like” odor and taste. Although the algal and microbial omega-3 LC-PUFA have somewhat enhanced stability and lesser organoleptic problems, these also are not easy to use in food products. Recently different methods have been devised to overcome the above problems. These include encapsulation of the omega-3 fatty acids in different food compatible matrices, as well as the use of special purification and distillation techniques and special antioxidants or antioxidants mixtures. Thus, the food products of the invention may be designed to include these beneficial dietary ingredients at adequate levels in a variety of daily food products.

Oxidative stress and other adverse effects related to oxidative damage have been shown to be controlled, at least to some extent, by different dietary ingredients with an anti-oxidation capacity. These can include plant extracts such as rosemary extract, lycopene, zeaxanthin, polyphenols (such as hydroxytyrosol found in olive oil, grapes, pomegranates, etc.), vitamins (tocopherols and especially vitamin E, ascorbic acid), minerals (Zinc, Selenium or Calcium), coenzyme Q₁₀ and beta carotene. All these ingredients, as well as other anti-oxidants, have been extensively investigated and shown to have beneficial effects on the health of individuals.

These antioxidants have also been used as dietary supplements. A few of these, especially vitamin E and ascorbic acid derivatives have been used at low levels (lower than 0.2% wt) in foods as food antioxidants and in some cases at even higher levels in order to deliver said antioxidants to exert their beneficial activity on the food consumers. No attempts have been made to use anti-oxidants in specialty diabetic food products as part of a diabetic functional food methodology addressing their CVD health problems, as suggested herein.

A variety of herbal or plant extracts, such as soy proteins, isoflavones, carotenoids (beta-carotene, lutein, zeaxanthin) and garlic extracts have been shown to beneficially influence different CVD risk factors such as high blood cholesterol level, high blood triglycerides level. No attempts have been made to use such plant extracts in specialty diabetic food products as part of a diabetic functional food methodology addressing their CVD health problems, as suggested herein.

Other ingredients or combinations of ingredients have also been shown to have beneficial effects and can be used as dietary ingredients in the food products of the invention. For example, a combination of vitamin B₆, vitamin B₁₂, and folic acid has been shown to lower blood level of homocysteine, high levels of the latter have been recognized as a CVD risk factor or bio-marker.

Additionally some dietary active ingredients with CVD related benefits have been shown to act synergistically to further reduce the risk of CVD. Such a combination includes blood cholesterols lowering ingredient phytosterols and soy proteins.

In one embodiment of the food article of the invention, said article is functional in the treatment and/or prevention of CVD risk factors, such as hyperlipidemia, which may be hypercholesterolemia and/or hypertriglyceridemia, oxidative stress and atherosclerosis, particularly in diabetic or diabetes-prone individuals.

Thus, the food article of the invention is geared to offer preventive or therapeutic health benefits, especially with regards to the cardiovascular health of subjects suffering from or prone to diabetes. As mentioned above, diabetes-prone subjects are individuals that fulfill at least one of the following criteria: (a) have a family history of diabetes mellitus (parents or siblings); (b) are obese; (c) belong to a race or ethnicity which have a higher incidence of diabetes, for example African American, Hispanic, Native American, etc.; (d) are 45 years old or older; (e) were previously identified with impaired fasting glucose (IFG) or impaired glucose tolerance (IGT); (f) have hyperinsulinaemia; (g) have hypertension, i.e. blood pressure above 140/90 mm Hg; (h) have dyslipidemia: HDL lower than 35 mg/dL and/or triglycerides above 250 mg/dL; (i) have a history of gestational diabetes or delivery of babies weighting above 4.1 kg.

A subject will be considered diabetic if his fasting plasma glucose is 126 mg/dl or over. Normal range of fasting plasma glucose is 60-109 mg/dl.

In another embodiment of the food article of the invention, said article further comprises at least one active agent which is functional in the treatment and/or prevention of GVD risk factors, such as hyperlipidemia including hypercholesterolemia and/or hypertriglyceridemia, metabolic disorders such as metabolic syndrome and obesity also considered diabetes related risk factors, oxidative stress and atherosclerosis. Said active agent may replace some or preferably all the fat constituents of the food product described in the invention.

The food product described herein should be considered beneficial to any condition that renders a subject, especially a diabetic or a per-diabetic individual, prone to acute CVD.

As herein described, said active agent is any one of phytosterols and/or phytostanols esters and their derivatives, diglycerides (DAG), combinations of phytosterol esters and DAG (specifically combinations having phytosterol-esters levels higher than DAG levels, optionally dissolved or dispersed in an oil and/or fat), anti-oxidants, natural herb and plant extracts (for example garlic extract, soy protein, soy isoflavones, or lycopene), omega-3 fatty acids, as well as other dietary ingredients or any combination thereof.

The anti-oxidant may include natural or synthetic anti-oxidants, such as polyphenols, vitamins, especially tocopherols, or minerals, such as zinc and selenium, known to exert positive and healthy effects on heart-health of the general population.

In addition, said active agent may include omega-3 lipids, in the form of free fatty acids, ethyl-esters, triglycerides, phospholipids or any other derivative or delivery platform, chemical or technical. These lipids have been shown to positively affect general heart-health indications in the general population as well as CVD prone population.

As mentioned above, the active ingredients can also be pharmaceutical in nature, such as statins, bile acid sequestrants, ezetimibe, blood diluting agents, and blood pressure lowering agents.

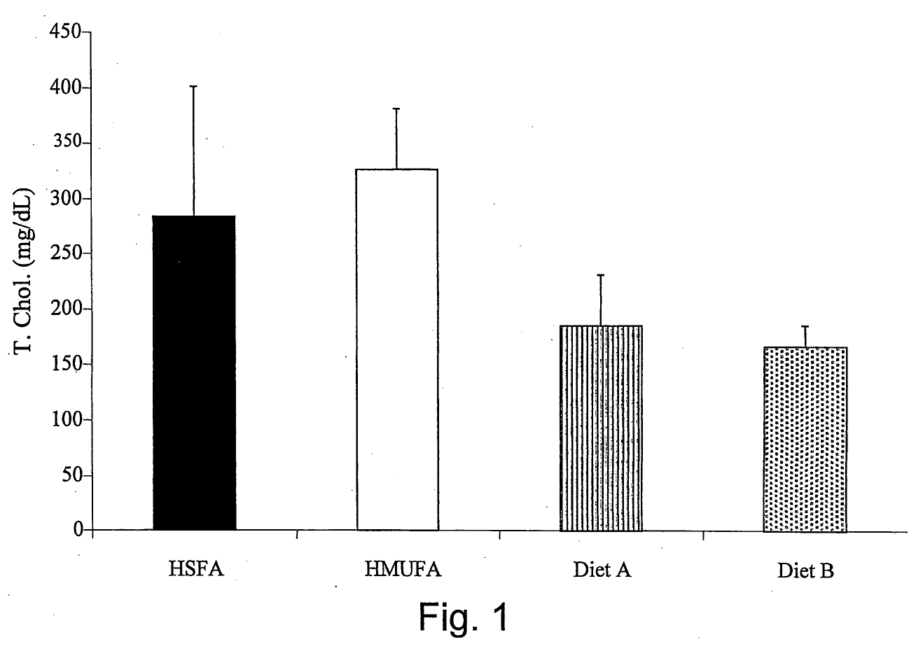

In one embodiment the active agent comprised in the food product of the invention may be any ingredient functional in reducing at least one of the following parameters: total cholesterol serum level, non-HDL cholesterol serum level, total cholesterol/HDL ratio and triglycerides serum level in an overweight and/or obese and/or diabetic subject and/or diabetic-prone subject, as illustrated in Examples 1 and 2, FIGS. 1-6.

High cholesterol in the blood is a major risk factor for heart and blood vessel diseases such as atherosclerosis and stroke. The term “total cholesterol” relates to three major kinds of cholesterol: High Density Lipoprotein (HDL), Low Density Lipoprotein (LDL), and Very Low Density Lipoprotein (VLDL). Blood total cholesterol values less than 200 mg/dL, and LDL Cholesterol of 100 mg/dL or less are considered optimal by the National Heart, Lung, and Blood Institute.

Excessive amount of LDL cholesterol in blood is deposited in the arteries, therefore, LDL level of less than 130 mg/dL is recommended and 100 mg/dL is considered optimal. LDL amount is commonly estimated by calculating its part from the total cholesterol, HDL, and triglycerides results (“LDL Calc”) or by a directly measurement.

HDL cholesterol is considered to be protective against heart disease by helping removing excess cholesterol deposited in the arteries. High HDL levels are associated with low incidence of coronary heart disease. Blood values of 35 mg/dL and higher are recommended.

The total cholesterol to HDL cholesterol ratio is a number that is helpful in predicting atherosclerosis and is validated as a powerful predictor for myocardial infraction (MI). The number is obtained by dividing total cholesterol by HDL cholesterol. (High ratios indicate higher risks of heart attacks, low ratios indicate lower risk). An average ratio is about 4.5 and a preferred ratio would be 2 or 3 or less than 4.

Triglycerides are the major storage form of fat in the body. In some people, abnormally high blood triglyceride levels (hypertriglyceridemia) are inherited, but it is often caused by non-genetic factors such as obesity, excessive alcohol intake, diabetes mellitus, kidney disease, and estrogen containing medications such as birth control pills.

High levels of triglycerides increase the risk of coronary heart disease (CHD) by speeding up plaque build-up on arteries (atherogenesis) and increasing the risk for thrombosis, which may lead to myocardial infarction. Desired triglycerides blood level range from 150-200 mg/dL.

High levels of triglycerides should be treated aggressively with low fat diets and, if needed, medications. The first step in treating hypertriglyceridemia is a low fat diet with a limited amount of sweets, regular aerobic exercise, loss of excess weight, reduction of alcohol consumption, and stopping cigarette smoking. In patients with diabetes mellitus, meticulous control of elevated blood glucose is also important. Therefore, the consumption of the nutritional product of the invention would be considered highly beneficiary.

If necessary, additional pharmaceutical agents such as fibrates (for example gemfibrozil), nicotinic acid, and statin derivatives can be added to the nutritional product. Said pharmaceutical agents, also may affect the overall lipid profile: Lopid not only decreases triglyceride levels but also increases HDL cholesterol levels and LDL cholesterol particle size; nicotinic acid lowers triglyceride levels, increases HDL cholesterol levels and the size of LDL cholesterol particles, as well as lowers the levels of Lp (a) cholesterol; statins are effective in decreasing triglyceride as well as LDL cholesterol levels and in elevating HDL cholesterol levels.

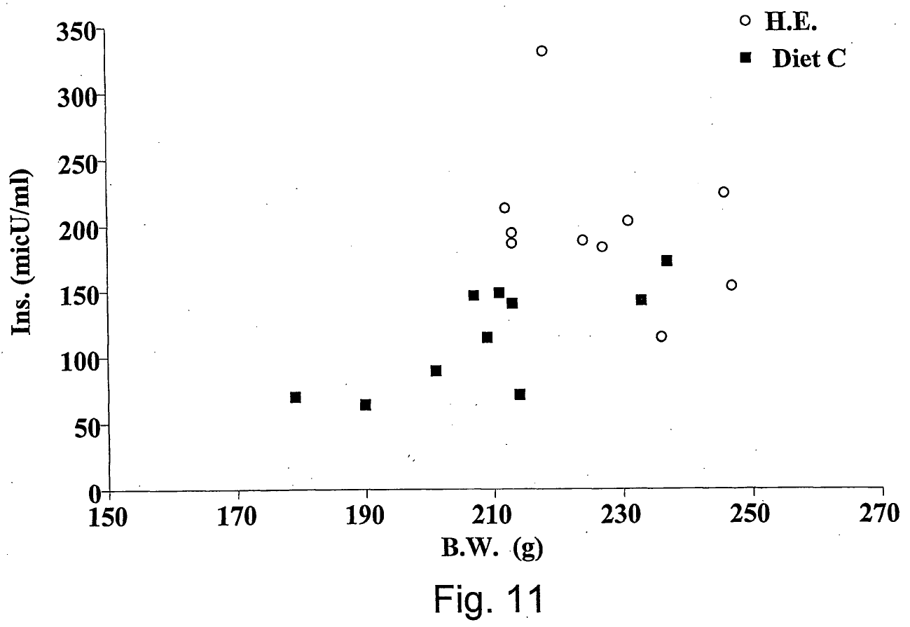

The food product of the invention is also functional in reducing the body weight and/or the serum insulin level in an obese and/or diabetic subject as illustrated in Example 2 and FIG. 11.

Insulin is a peptide hormone that enables the body to metabolize and utilize blood glucose by permitting the cells glucose uptake and lowering its concentration in blood. Inside the cell, glucose is either used for energy or stored in the form fat. Insulin drives to use more carbohydrate, and less fat, promoting to fat metabolic imbalance and obesity, considered risk conditions to develop diabetes and CVD. Therefore, it is desirable to control the blood insulin level whenever it overpasses normal values. Normal fasting insulin values range 5-20 mcU/mL (micro unit per milliliter).

Particularly, said food product is functional in ameliorating hyperinsulinemia (reducing the serum insulin level) in an insulin-resistant subject. High insulin blood level could be used as an indicator for insulin resistance, one of the main leading conditions for developing Type 2 diabetes, and usually occurring in individuals suffering from hypertension, cardiovascular disease, and obesity. Usually, the glucose/insulin ratio (G/I ratio) index is used for the diagnosis of insulin resistance, which low values depict higher degree of insulin resistance. A desired G/I ratio will be of less than 4.5.

Since the food product of the invention reduces the serum insulin level, measurement of the G/I ratio may be used as a simple test for evaluating the therapeutical effectiveness of the said food product

In a more particular case, said food product is effective in ameliorating hyperinsulinema or preventing progression of insulin resistance in an obese subject prone to develop diabetes. Physical activity and weight loss help the body the better respond to insulin and overcome insulin resistance. Body weight reduction accomplished by consuming the nutritional product of the invention and being physically active, may prevent the evolution of the insulin resistance condition into developing Type 2 diabetes.

By changing nutritional habits and loosing weight it is possible to return pre-diabetic individuals to normal blood glucose levels and reduced the risk of diabetes by 58 percent.

In another embodiment, the food product of the invention is functional in reducing the body weight, inhibiting body weight gain and reducing insulin resistance in obese, subjects with metabolic imbalances such as diabetes or metabolic syndrome, or in an individual prone to develop diabetes or obesity.

Furthermore, these active ingredients may assist to suppress WAT accumulation, remodel body fat distribution and reduce visceral fat weight, by shifting fats accumulation, from the visceral tissues to the peripheral tissues, resulting in prevention or inhibition of metabolic syndromes in obese, overweight, diabetic, pre-diabetic, or in an individual prone to diabetes or obesity, or by simply ameliorating diabetic patients' conditions.

Dietary ingredients that address weight gain and management as well as fat distribution include diglycerides (DAG). Replacement of daily fat intake with DAG has been shown to promote weight loss. Additionally, conjugated linolenic acid (CLA) has also been extensively researched and connected to health benefits related to weight management. Recently, novel triglycerides alkyl substituted at the alpha position of their fatty acids have been shown to inhibit digestion lipases and promote satiation feeling resulting in overall weight loss. Such dietary ingredients, as well as other ingredients addressing weight gain, weight management and controlling fat distribution are all within the scope of this invention.

Untreated diabetes might evolve to develop a variety of secondary disease complications. The food product of the invention is intended to be use for reducing the risk of life threatening long term diabetes complications and ameliorate the deterioration of life quality in an obese and/or diabetic subject.

Diabetes complications are classified in three major categories macrovascular, microvascular and of neurological origin. People with diabetes usually develop heart and blood vessel disease. Diabetes carries an increased risk for heart attack, stroke, and complications related to poor circulation.

Other diabetes complications may include kidney diseases, eye problems that may lead to blindness, diabetic neuropathy and nerve damage, many different foot problems resulting from nerve damage or poor blood flow that may conduct to amputations, skin disorders which sometimes is the first sign that a person has diabetes, gastroparesis and depression.

Therefore, the food product of the invention should be considered to be used for the treatment and/or prevention of any one of the diabetes complications selected from the group of retinopathy, nephropathy, diseases of the large vessels supplying the legs dower extremity arterial disease), coronary heart or artery diseases, cerebrovascular diseases and disturbed neural function afflictions, or any other condition which may lead to blindness, end-stage kidney disease (ESRD) and amputations, myocardial infractions, stroke or any other complication mentioned above.

In an additional embodiment, the food product of the invention further comprises active agents, dietary or pharmaceutical ingredients, for addressing other diabetes related problems. The active agent of said food product which is functional in the prevention and/or treatment of acute cognitive decline is any one of phosphatidylserine, ginko biloba, brahmi (Bacopa monneri), and omega-3 containing fat and said acute cognitive decline may be associated or induced by diabetes.

Several cognitive related dietary ingredients can be used in the food products of the invention. Phosphatidylserine, a major brain phospholipid mainly produced from soy phospholipids, has been previously and extensively shown to improve memory and other cognitive functions in the elderly and in younger populations. Several herbal extracts, such as ginko biloba, have also been claimed to have similar cognitive benefits. Omega-3 fatty acids and antioxidants, which both have been connected to the promotion of heart health, have also benefits on cognitive functions. Antioxidants are connected to brain health since many cognitive decline processes are the result of oxidative damage to brain cells and tissues.

In a further embodiment of the invention, the food product further comprises active agents which are functional in the prevention and/or treatment of acute eye conditions associated with Type 2 diabetes, wherein said active agents are vitamins, antioxidants, and other natural eye-health promoting ingredients or extracts.

The amount of the dietary or pharmaceutical ingredients which proactively promote the health of the heart, brain, and eye, that is in the food products of the invention can be based on their specific RDAs or other recommendations of different organizations or based on the results of related clinical trials. Each ingredient can be given at its full RDA per serving or alternatively only part of the RDA can be given in each serving, depending on the variety of foods or sources available for daily consumption that deliver the same ingredient or the average number of servings usually consumed from the specific food product.

The guiding rule of the invention is that the active ingredient could be consumed at an adequate level to exert its beneficial health effects through one or more food products and/or servings, either once a day or through different meals during the day. At least 5% of the recommended, official or non-official, daily intake of said ingredients should be provided by the food products of the invention.

The active dietary or pharmaceutical ingredients may be added at any stage during the food product preparation or processing and either through its major mass or through fillings, coatings, etc.

The active dietary or pharmaceutical ingredients may also exert technical or functional properties to the food product, such as related to texture, stability, shelf-life, organoleptic and sensorial qualities and appearance.

As described herein, the food product of the invention is preferably for use by any subject either suffering from diabetes or prone to become diabetic. Such food articles can be consumed by diabetics, as part of their unique diets, for treating or addressing their cardiovascular risk factors. Optionally these foods can be used by pre-diabetic individuals in order to prevent or inhibit the manifestation of diabetes.

The food product described herein may be any one of dairy products, bakery products, condiments, beverages and drinks, snacks, candies, ice-creams and frozen desserts, morning cereals, nutrition bars, chocolate products, prepared foods, grain products and pasta, soups, sauces and dressings, confectionery products, oils and fats products, dairy and milk drinks, soy milk and soy dairy-like products, frozen food products, prepared meals and meal replacements, meat products, cheeses, yoghurts, breads and rolls, yeast products, cakes and cookies and crackers.

The present invention is defined by the claims, the contents of which are to be read as included within the disclosure of the specification.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### EXAMPLES

### In Vivo Experiments

The effect of different dietary functional oils and lipids on CVD was estimated by measuring some parameters known as CVD risk factors (body and fat weight, blood lipid levels, glucose and insulin blood levels) in Psammomys obesus, an experimental animal model for nutrition-dependent type 2 diabetes. Psammomys obesus, an Israeli sand rat, is a herbaceous gerbil that when maintained in captivity and given free access to non-purified high energy (HE) rodent diet tends to display heterogeneous glucose and insulin levels, ranging from normoglycemia and normoinsulinemia to obese diabetic animals with progressive hyperglycemia and hyperinsulinemia. The course of the obesity and diabetes developed in these animals can be characterized in four phenotypic states: normoinsulinemia and normoglycemia (State A), hyperinsulinemia but normoglycemia (State B), hyperinsulinemia and hyperglycemia (State C), and hypoinsulinemia and severe hyperglycemia as a result of β-cell degranulation, and markedly reduced pancreatic insulin content (State D). The Psammomys obesus gerbil animal model has been shown in numerous studies to be an ideal natural model of Type 2 diabetes disease in humans because it demonstrates an increased tendency to develop diet-induced diabetes, which is associated with moderate obesity. This model has been used also to study the prediabetic state, in order to determine the development of metabolic changes such as hyperinsulinemia, hyperglycemia, and obesity, leading to diabetes [Zimmet P. (2001) Nature 414:782-7, review by Ziv E. and Kalman R. (2001), In Animal models of diabetes, eds. Sima and Shafrir, Harwood academic publishers, 327-342]].

In humans, as well as in the Psammomys obesus model, diabetes is associated with obesity and nutritional values and characteristics of their diets are of importance in the onset and evolution of the disease (Shafrir E. and Gutman A. (1993) J Basic Clin Physiol Pharmacol. 4: 83-99; Kalman R. et al. (1993) J Basic Clin Physiol Pharmacol. 4: 57-68). The selection of the Psammomys obesus as the present experimental model, contributes to the understanding of the role that dietary functional oils and lipids play in the development and/or treatment of obesity and non-insulin dependent diabetes mellitus.

The gerbil experimental model is known to be slightly superior for assessing blood lipid-lowering effects as compared to hamster models. Rats and mice are considered inappropriate because their plasma and liver cholesterol is relatively less responsive to dietary cholesterol challenge. In the Psammomys obesus model a sequential transition from normal to impaired insulin sensitivity accompanied by increased adiposity prior to insulin resistance and obesity occurs in a manner similar to that observed in the human Type 2 diabetes susceptible populations.

### Materials and Methods

The animal studies were approved by the Institutional Animal Care and Use Committee of Hebrew University and the Hadassah Medical Organization.

Animals: Male Psammomys obesus gerbils (age 2.0-3.5 months) obtained from Harlan Laboratories Ltd. (Jerusalem, Israel) were grown at the Hebrew University facilities. After weaning period, Psammomys obesus were maintained on a low-energy diet containing 2.38 kcal/g (Koffolk Ltd., Petach Tikva, Israel) prior to diabetes induction.

### Experimental Model—I

Psammomys obesus were switched to a high energy diet containing 2.93 kcal/g (Tekled Global, Madison, Wis., USA) for 4 weeks, and the animals that developed diabetes (˜70% of the animals in the Psammomys obesus colony) were selected for the feeding intervention study. Animals were considered diabetic if their non-fasting blood glucose was greater than 180 mg/dL.

Diabetic gerbils were randomly assigned to groups (8-10 animals per group) and fed for another 3.5 weeks with the different high energy experimental diets, as specified in Table 2. The high fat diets supplied differ in their fat content. Water and food were supplied ad libitum; Psammomys obesus blood glucose concentrations and body weights were monitored every other day. By the end of the experiment, the gerbils were anesthetized with ketamine (Ketalar; Parke-Davis & Co., Gwent, United Kingdom) and exsanguinated by cardiac puncture. Blood samples were collected into an EDTA-wetted syringe and used for biochemical analyses.

### Experimental Model—II

Sixty adult male Psammomys obesus gerbils were randomly assigned to 2 high energy diets containing 2.93 kcal/g (n=30), that differed only in the fats and lipids content; either Harlan-high energy diet (Tekled Global, Madison, Wis., USA) or diet C (Table 2). Water and food were supplied ad libitum for 4.5 weeks; body weights and blood glucose concentrations were monitored every other day. After 4.5 wk of feeding with the experimental diets, gerbils were deprived of food overnight (16 h) and anesthetized with ketamine (Ketalar; Parke-Davis & Co., Gwent, United Kingdom) and exsanguinated by cardiac puncture, and blood samples were collected into an EDTA-wetted syringe. The liver and the epididymis fat were excised and weighed. Blood taken from the heart was used for biochemical analyses.

Glucose analysis Blood glucose concentration was determined by the enzymatic glucose analyzer, Glucometer Elite (Bayer, Elkhart, Ind., USA) on blood samples taken from the tail vein.

Lipid analysis Plasma samples obtained from EDTA-treated blood samples (separated at 12,000×g for 15 min) were analyzed for total cholesterol, total triglycerides and HDL-cholesterol levels by colorimetric methods (Boehringer Mannheim, Mannheim, Germany). LDL-cholesterol level was calculated using the Friedewald equation.

Insulin analysis Insulin levels were assessed by radioimmunoassay using a human primary antibody (Phadesph; Xabi Pharmacia Diagnostics, Uppsala, Sweden).

Statistical Analysis Data was analyzed by one-way analysis of variance (ANOVA) to assess the differences among the experimental groups. Differences between mean values were evaluated by the Student's two tailed t-test.

Nutritional Diets Standard milling procedures were used to incorporate different treatment oils (custom-made manufactured by Harlan Tekled Ltd, USA) into standard gerbil chow. The 2018SC+F high-energy diet was used as a control (Tekled Global, Madison, Wis., USA).

The composition of the custom-made diets was similar with respect to the food and nutrient content. All diets were made from the same basal mix while the variable component was the supplemented fats. This basal mix was a slightly concentrated version of 2018SC+F without a fat source. When the fat source and cholesterol were added, the finished products were very similar to the original 2018SC+F, with the exception of the fat and cholesterol components.

Diets were prepared according to the scheme below:

Basal Mix

| | |
|---|---|
| Protein | 23.6% |
| Fats | 2.4% |
| Carbohydrate | 68% |
| Ash + Mineral + vitamins | 6.0% |

TABLE 1
Fatty acids composition of supplemented
oils (% of total fatty acids)

| | Soybean | | | Preparation | Preparation |
|---|---|---|---|---|---|
| | oil | SFA oil | HOSO oil | A | B |
| C14 | | 0.7% | | | |
| C16 | 11% | 37% | 4% | 3.6% | 5.7% |
| C18 | 3.6% | 4.9% | 3.6% | 3.3% | 3.2% |
| C18:1 | 23% | 25% | 81% | 84.7% | 20.2% |
| C18:2 | 54% | 29% | 11% | 8.4% | 15.1% |
| C18:3 | 6.4% | 3.3% | | | 55.8% |

Abbreviation: SFA = saturated fatty acids; HOSO = high oleic sunflower oil

TABLE 2
Diet Compositions

| | High | | | | | |
|---|---|---|---|---|---|---|
| | energy | HSFA¹ | HMUFA² | Diet A | Diet B | Diet C |
| | (g/Kg) | (g/Kg) | (g/Kg) | (g/Kg) | (g/Kg) | (g/Kg) |
| Basal mixture | 938.5 | 938.5 | 938.5 | 938.5 | 938.5 | 938.5 |
| Ground Corn | 20.0 | | 20.0 | 20.0 | 20.0 | 20.0 |
| Cholesterol | | 1.5 | 1.5 | 1.5 | 1.5 | |
| Soybean oil | 40.0 | | | | | |
| SFA oil | | 60.0 | | | | |
| HOSO oil | | | 40.0 | | | |
| Preparation A³ | | | | 40.0 | | 40.0 |
| Preparation B³ | | | | | 40.0 | |
| % Fats | 6 | 8 | 6 | 6 | 6 | 6 |
| % cholesterol | 0 | 0.15⁴ | 0.15⁴ | 0.15⁴ | 0.15⁴ | 0 |

¹HSFA = Highly enriched with saturated fatty acids oil
²HMUFA = Highly enriched with mono-unaturated fatty acids oil, i.e. HOSO
³Contain 20% (w/w) plant sterols esterified to indicated base oil derived fatty acids and 15% (w/w) dietary diacylglycerol presenting similar fatty acids composition as base oil
⁴Dietary plant sterol to cholesterol ratio of 5:1

### Example 1

### Effect of Mono- and Poly-Unsaturated Fatty Acid Diets on Diabetic Gerbils' Blood Lipids Content

The present experiment studies the effect of consumption of Diet A (PS-E+DAG, comprising soy sterols esters with HOSO oil (PS-E) and diacylglycerols derived from HOSO oil (DAG)) and Diet B (PS-E+DAG, comprising soy sterol esters with flaxseed oil (PS-E) and diacylglycerols derived from flax seed oil (DAG), having high content of polyunsaturated fatty acids) on the profiles of plasma lipids.

Diabetic male Psammomys obesus gerbils were assigned randomly to the indicated diet groups (8-10 gerbils each) for 3.5 weeks feeding period (as presented in Table 1 and Table 2). By the end of the experimental feeding period, blood samples were collected for plasma lipids analyses.

The total cholesterol plasma level in the different diet groups significantly varied as illustrated in FIG. 1. A striking difference can be observed among the controls and the diet-treated groups (diets A and B), yet all animals maintained similar basal levels of hyperglycemia and hyperinsulinemia symptoms. Diabetic HMUFA-fed and HSFA-fed Psammomys obesus showed similar total cholesterol concentrations in plasma (P-value=0.24), while the other Psammomys obesus groups which were fed with diet A and diet B had a substantial and significant reduction in the levels of total cholesterol (35% and 42%, respectively; P-value=0.031, 0.028) relative to saturated fat fed gerbils.

The high statistical significance of these results (shown by ANOVA, P-value=0.003) suggest that both preparations A and B, induce a very potent hypocholesterolemic effect. Similar results (˜43% reduction) of plasma total cholesterol reduction were reported for male Mongolian gerbils (Meriones unquiculatus) frequently fed with 5:1 phytosterol to dietary cholesterol for four weeks [Hayes et al. (2002) J. Nutr. 132: 1983-1988].

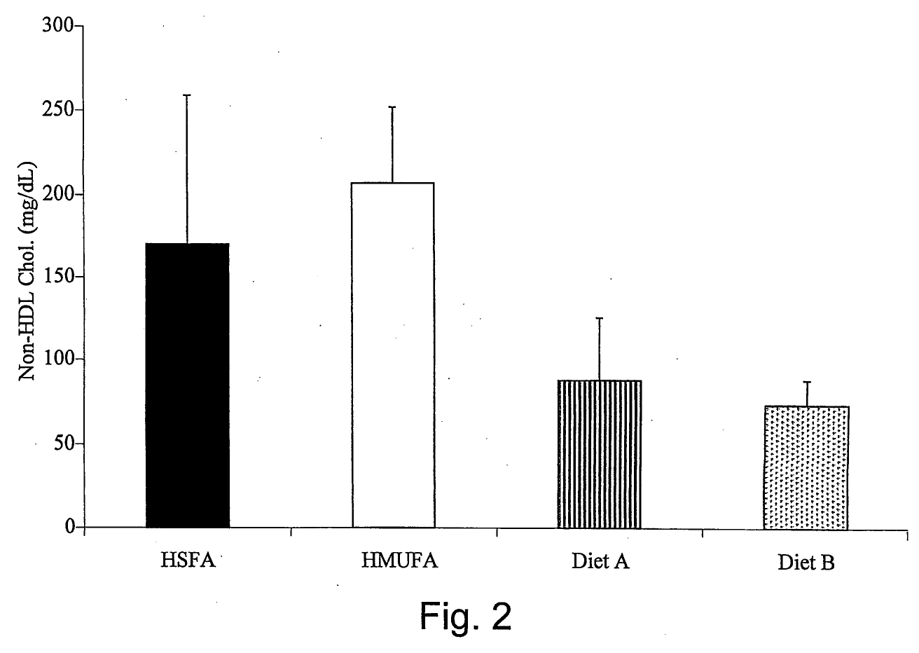

Similarly, as shown in FIG. 2, consumption of either diet A or diet B induced a remarkable and significant reduction in the level of plasma non-HDL cholesterol (47% and 59%, respectively; P-value=0.022, 0.019) relatively to diabetic gerbils fed with HSFA diet. In the HSFA and UFA diet consumption groups (controls), similar non-HDL cholesterol concentrations were measured (P-value=0.22). ANOVA comparison between these diets matrices suggested a highly significant difference (P-value=0.001).

Non-HDL-C serum level is considered a strong predictor of future risk for cardiovascular complication among patients which not necessarily exhibit any vascular disease symptom. Non-HDL-C was recently recommended by the National Cholesterol Education Program, Adult Treatment Panel III as a secondary treatment target (after LDL-C) in patients with elevated triglycerides.

Both FIG. 1 and FIG. 2 highlight the prominent hypocholesterolemic effect of either preparation A or preparation B of the invention inasmuch as both resulted in reduced total and non-HDL cholesterol plasma levels; in comparison to consumption of diets based on either saturated fatty acids or high oleic sunflower oils. Contrarily, no significant difference was observed in the plasma HDL-cholesterol level of any of the gerbils tested groups (ANOVA P-Value=0.11).

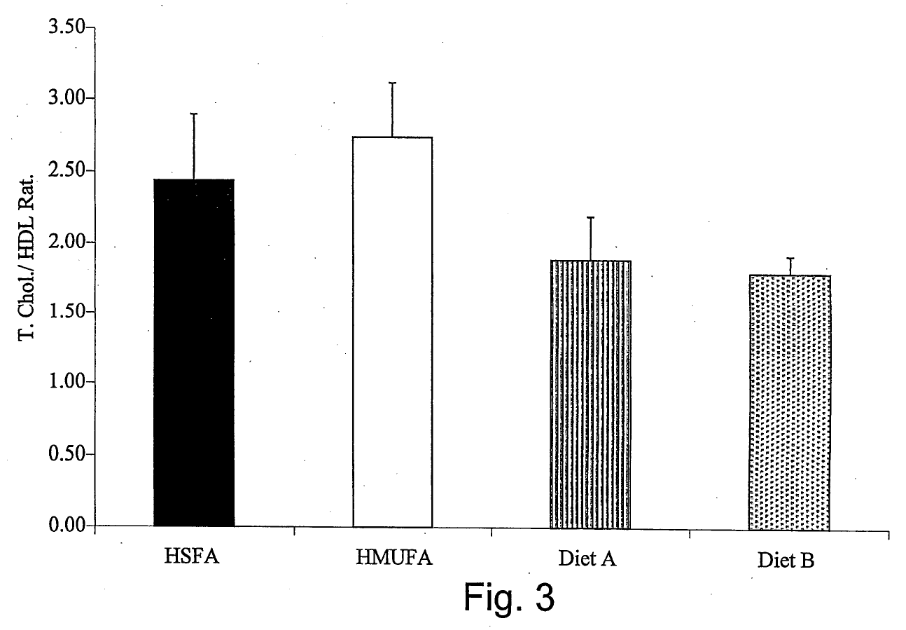

The calculated total cholesterol to HDL-cholesterol ratio (Total/HDL) illustrated in FIG. 3 and further statistically analyzed (ANOVA P=0.0005) showed a significant difference between the different dietary treatment groups. The Total/HDL ratio in the HMUFA diet consumption group was comparable to the ratio seen in the HSFA-fed obese diabetic Psammomys obesus (P-value=0.14). In spite of these results, when diet A or diet B of the invention were provided to these sand rats, a remarkable and significant decreased (23%; P-Value<0.012, and 27%; P-Value<0.007) in the Total/HDL cholesterol ratio was measured relatively to the control group (HSFA-fed group).

In Type 2 diabetic patients dyslipidemia is manifested by elevated triglycerides level and reduced HDL cholesterol level. Usually, the LDL cholesterol amount does not significantly differ in Type 2 diabetic patients and non-diabetic individuals, but some LDL appears as a denser particle form (apolipoprotein B) which may, increase the atherogenic risk despite the total and LDL cholesterol normal values.

In some cases, diabetic patients may have elevated levels of non-HDL cholesterol (LDL plus VLDL cholesterol). The ability of the nutritional components of diets A and B to reduce the non-HDL level in such a short period of time is of major relevance in reducing the risk, preventing and treating CVD. The use of the A and B food diet matrices should be considered as highly beneficial for dyslipidemia and LDL-cholesterol related atherogenicity treatment, together or independently of other conventional pharmaceutical drug treatments.

The dietary treatment of obese diabetic Psammomys obesus either with diet A (soy sterols esterified to long chain monounsaturated fatty acids in a diacylglycerol HOSO matrix) or diet B (soy sterols esterified to long chain polyunsaturated fatty acids in a diacylglycerol flax seed oil matrix) resulted in a significant reduction of the total cholesterol and non-HDL cholesterol values, despite said diets were supplemented with cholesterol. These results reveal a new non-pharmaceutical, non-synthetic and not-needing physical intervention tactic for changing the body lipid management in a short period of time, by a simple nutritional habit adjustment which could be of benefit to any diabetic, obese, or subject at risk of developing any high cholesterol-related complication.

The success efficiency of the nutritional product of the invention is based on the its special composition of low or glucose free content, balance fat content and the addition of a proactive agent (pharmaceutical or nutritional) for addressing the needs of diabetics or diabetic-prone individuals. Although, each one of the separated components may have some mild influence on the lipid profile, the synergistic activity of the components, as well as their relative ratios in the food product of the present invention are the reasons for making this product so effective in the treatment of lipid imbalanced conditions associated with diabetes.

### Example 2

### Effect of Monounsaturated Fatty Acids Diet on “Pre-Diabetic” Gerbils

Pre-diabetic individuals may exhibit high total cholesterol, LDL cholesterol, and triglycerides levels but low HDL cholesterol level as compared to non-diabetic individuals. This experiment comes to evaluate the effect of diet C, a cholesterol free diet, on the lipids blood level and lipids management in diabetic prone gerbils.

Sixty adult male Psammomys obesus gerbils were randomly assigned to two different high energy diets (n=30), which contained 2.93 kcal/g and only differed in their fat and lipids content. The effect of diet C (Table 2) was compared to the effect of the basic Harlan-high energy diet (Tekled Global, Madison, Wis., USA). After 4.5 weeks of experimental diet feeding, the gerbils were deprived of food overnight (16 h) and sacrificed. The liver and epididymal fat were harvested and weighed. Collected blood samples were used for biochemical analyses.

The effect of diet C on the development of obesity and diabetes in Psammomys obesus (an experimental model of nutritionally induced diabetes type 2), was compared to the rodent standard high energy (HE) diet.

According to pervious publications [Ziv and Kalman (2001), in Animal Models of Diabetes, eds. Sima and Shafrir, Harwood academic publishers, 327-342; Kalman R. et al. (1993) J Basic Clin Physiol Pharmacol. 4: 57-68], hyperglycemia is developed relatively fast, within 7-14 days of HE diet intake. Usually, gerbils will die after 40-70 days of HE diet, as consequence of D-cell function loss (phase D). In our study, by the end of the feeding period there was a slightly lower percentage, however typical, of diabetic animals in the diet C-fed group (73.3%) compared with HE diet (80.0%). This indication was further correlated with higher survival rate of the Psammomys obesus that consumed diet C (97%), whereas gerbils consecutively fed with HE diet demonstrated a somewhat lower survival rate (87%) by the day the animals were sacrificed, 4.5 weeks after the beginning of the feeding experiment.

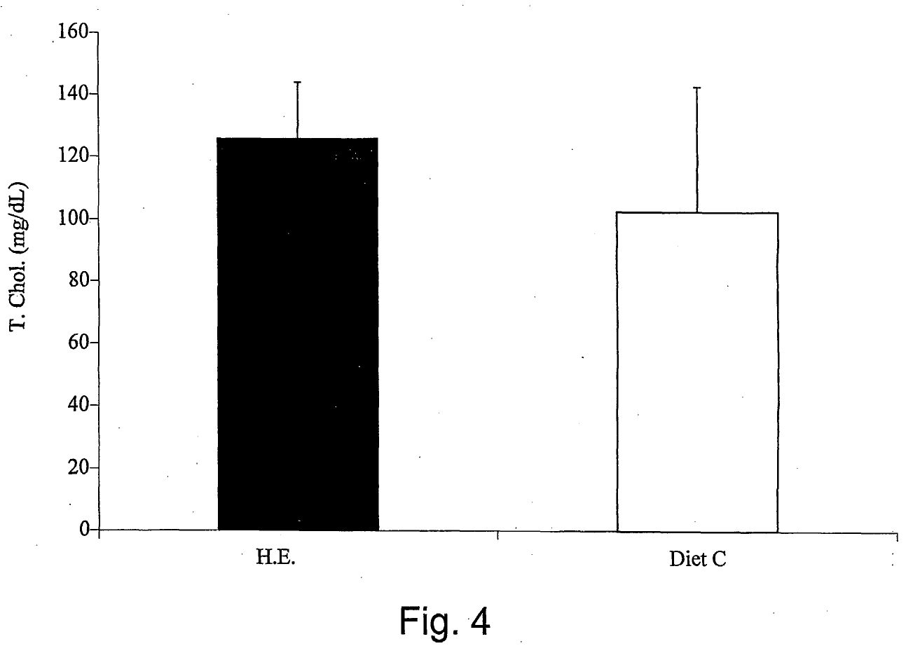

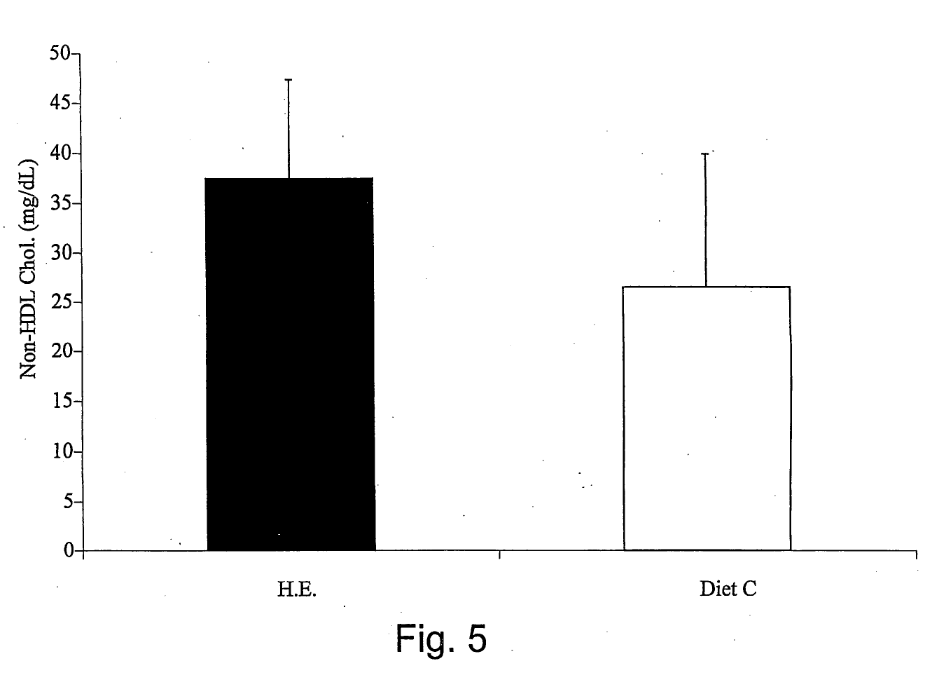

Consumption of diet C induced a major reduction of the total cholesterol level, compared with HE diet (23% decrease with P-value=0.055 as seen in FIG. 4). A more prominent and statistically significant reduction was observed in the non-HDL cholesterol level (41% decrease with P-value=0.026 as seen in FIG. 5).

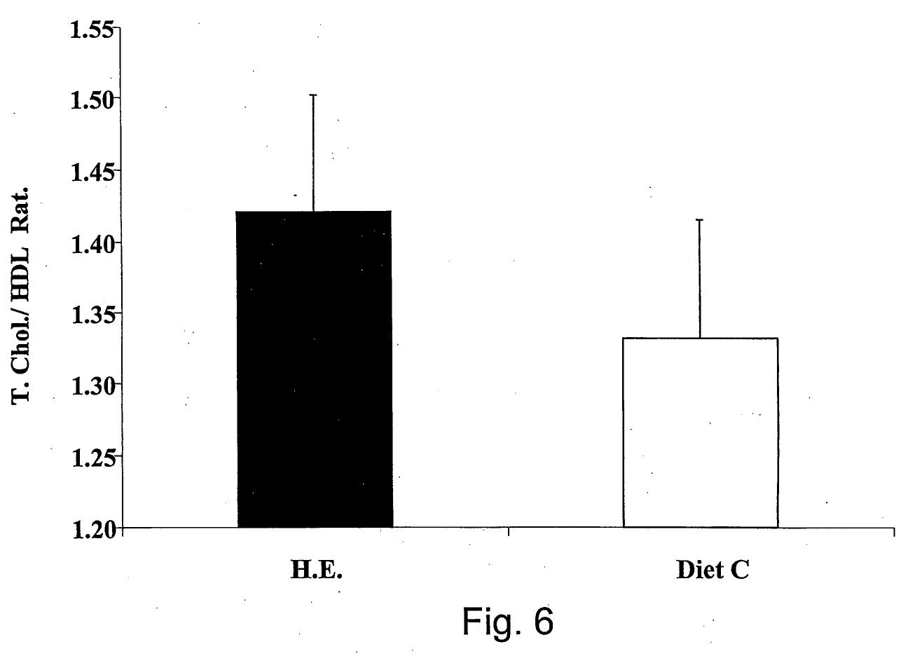

A small though highly significant reduction in the total cholesterol/HDL ratio was observed among gerbils fed with diet C as compared to HE fed gerbils (7% decrease with P-value=0.012 as seen in FIG. 6). This diminution could not be attributed to a parallel reduction in the HDL cholesterol level, since only a mild tendency was perceived when these two diets were compared (P-value=0.10).

As both diet C and HE contained no dietary cholesterol supplementation, it can be concluded that the efficacy of specialty food products containing proactive ingredients such as preparation A (see Example 17) is manifested by actually lowering total cholesterol both dietary and endogenous cholesterol, and especially LDL-cholesterol.

The dietary diacylglycerol content in diet C had no effect on the gerbils' triglycerides plasma level, as it was expected accordingly to previous reports [Murase et al. (2001) J. Lipid Res. 42:372-378; Murase et al. (2002) J. Lipid Res. 43:1312-1319].

Obesity and the metabolic disorder, which are recognized as risk factors for non-insulin dependent diabetes mellitus (NIDDM) and insulin resistance [Matsuzawa et al. (1995) Ann N Y Acad Sci 748:399-406], are identified as being the outcome of a disequilibrium between energy uptake and energy expenditure.

Increased body fat mass is frequently accompanied by elevated circulating free fatty acids, insulin, tumor necrosis factor-α, and leptin, suggesting that these molecules may play important roles in the development of insulin resistance in obese individuals. In order to elucidate the connection between obesity and insulin resistance some of the following anthropometric parameters were evaluated.

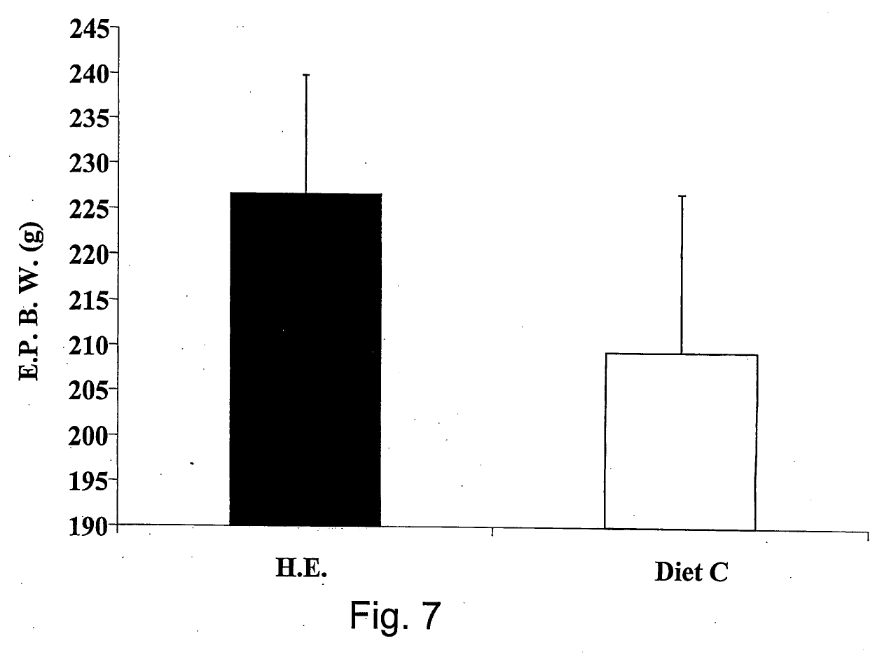

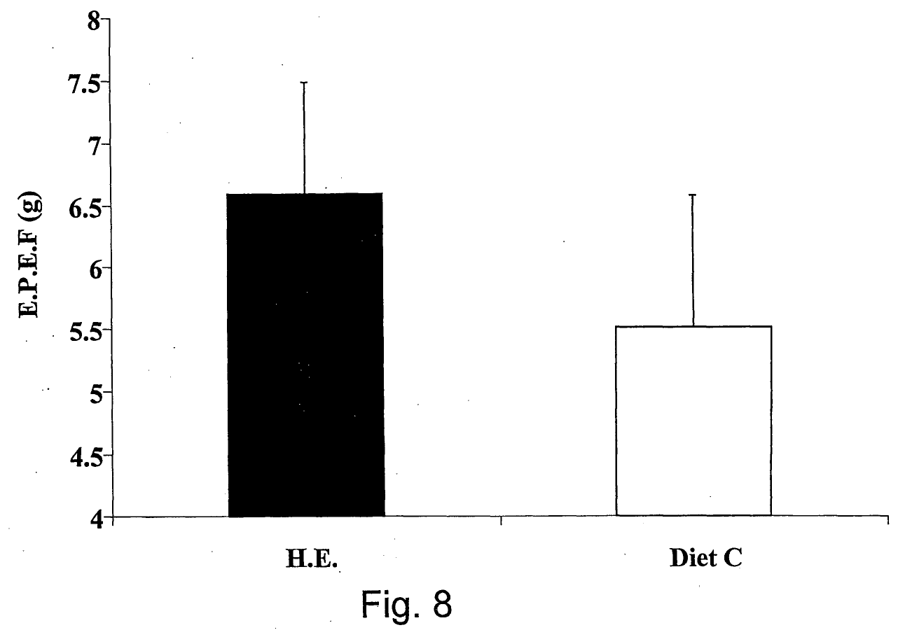

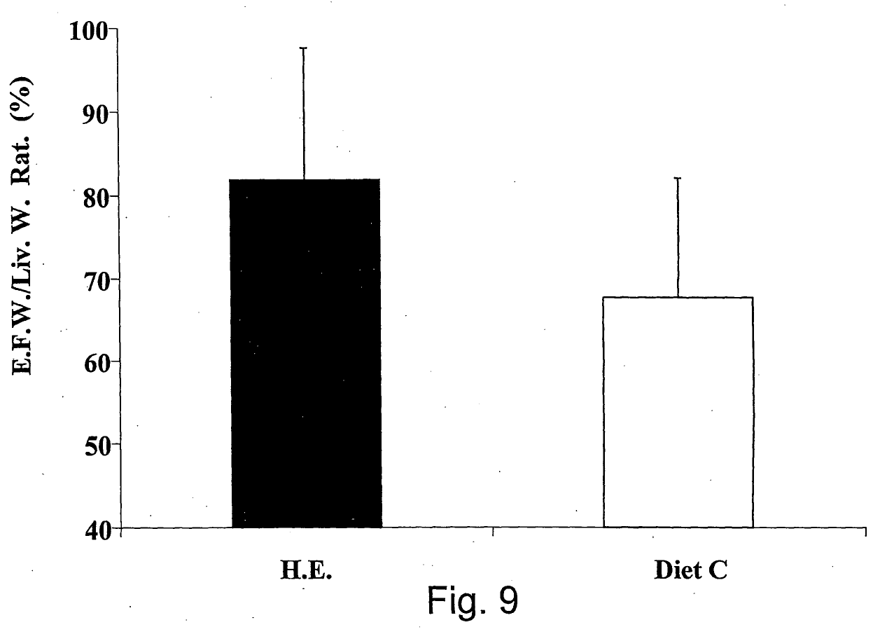

Consumption of diet C resulted in a marked reduced endpoint bodyweight as compared to HE fed gerbils (8.3% decrease with P-value=0.011 as seen in FIG. 7). Also, the epididymal white adipose tissue (WAT) weight, but not the liver weight (P-value=0.41), was remarkably reduced in diet C fed gerbils as compared to the HE control group (20% decrease with P-value=0.011 as seen in FIG. 8). A highly significant correlation of reduced relative size of organs expressed by the epididymal WAT to liver weight ratio is illustrated in FIG. 9 (21% decrease, P-value=0.025).

According to these results, it can be concluded that food products of the invention containing proactive ingredients such as preparation A not only affect the serum lipid profile but are also involved in the body fat accumulation and distribution.

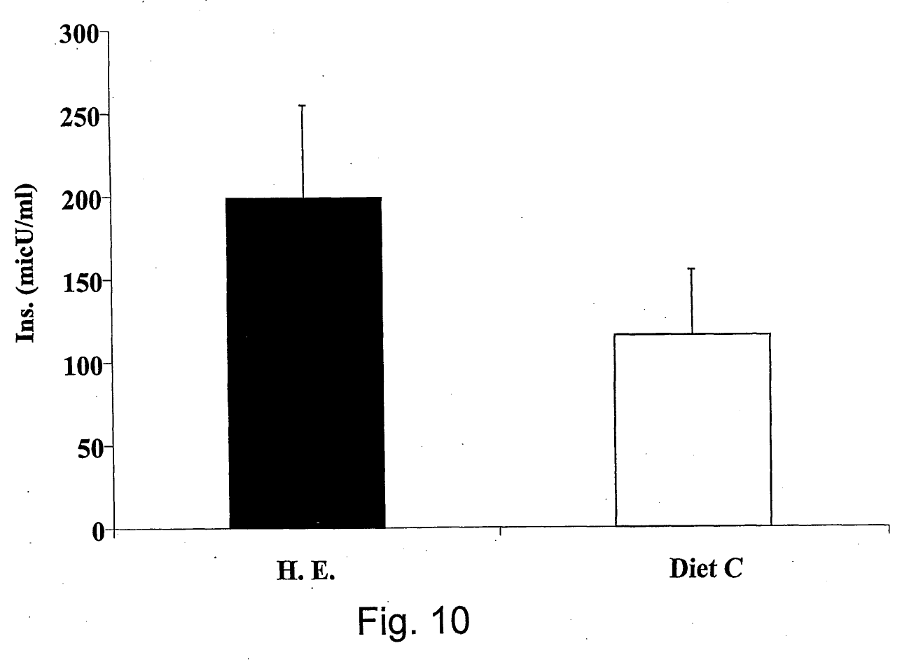

Diet C showed a significant effect on fasting plasma insulin levels as compared to the control HE group (42% decrease with P-value=0.0006 as seen in FIG. 10). Under fasting conditions, Psammomys obesus revealed a less pronounced hyperinsulinemia though the overall hyperglycemia remained similar (P-value=0.25).

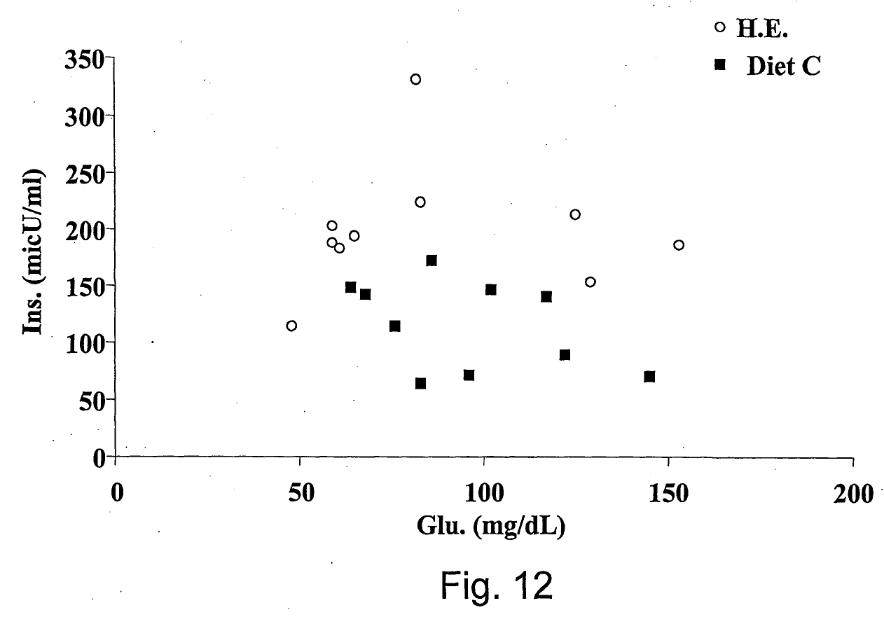

As expressed in FIGS. 11 and 12, gerbils fed with diet C presented a lower serum insulin/bodyweight index and a lower serum insulin/blood glucose level index as compared to HE fed gerbils. Therefore, for similar bodyweight or glucose level, animals fed with diet C showed a lower plasma insulin level (38% decrease with P-value=0.0015 and 49% decrease with P-value=0.0015 respectively).

The importance of the fasting insulin reduction should be looked in the context of reduced fat tissue accumulation and combined roll in insulin resistance propagation. The capacity of diet C and correspondingly food products containing preparation A to invert the metabolic imbalanced situation, has proven its beneficiary effects and effectiveness which should be applied for the prevention and treatment of diabetes-prone individuals, diabetic patients at different stages and other individuals suffering from other lipid imbalanced afflictions.

The proposed simple care which can be achieved by the use of the food products of the invention, such as products containing preparation A, is expected to be of great profit for inflicted individuals, since its beneficiary results are evident after a relatively short period.

### Example 3

### Yoghurt Drink with Phytosterols

The fruit flavored yoghurt drink of the invention is designed primarily for the diabetic and pre-diabetic populations and it includes no sugar, has a low fat content as to not contribute to the hypertriglyceridemia and/or hypercholesterolemia associated with diabetic conditions. Furthermore, the yoghurt drink is fortified with soy phytosterols, providing 100% of their RDA, in order to further lower high blood cholesterol levels.

Ingredients: pasteurized milk, maltitol, fruit puree/concentrate, milk powder, processed starches (E1422, E1442), natural stabilizer (E440), flavor agents, citric acid, soy phytosterols.

Nutritional values (100 ml): Protein 3.1 g, Carbohydrates 16.4 g, Fat 1.5 g (1.18 g milk fat, 0.32 g soy phytosterols), Calcium 110 mg, Sodium 76 mg. Each 250 ml serving contains 0.8 g of soy phytosterols (100% RDA).

### Example 4

### Soy Drink with Soy Isoflavones and Proteins

The natural flavored soy drink of the invention is designed primarily for the diabetic and pre-diabetic populations and it includes no sugar, has a low fat content as to not contribute to the hypertriglyceridemia and/or hypercholesterolemia associated with diabetic conditions. Furthermore, the soy drink is fortified with soy isoflavones and proteins, in order to further lower high blood cholesterol and LDL cholesterol levels. Isoflavones can preserve the vascular elasticity and inhibit atherosclerotic cascades. Soy proteins have also been shown to have a cardiovascular protective effect and may reduce the risk to heart disease.

Ingredients: water, soy beans, artificial sweeteners, poly alcohols, calcium fortification, acidity regulators (E-339, E-452), salt, flavor, stabilizer (E-407), soy isoflavones.

Nutritional values (100 ml): Protein 3.5 g, Carbohydrates 3.9 g, Fat 1 g (of which 10% saturated), Calcium 150 mg, Sodium 50 mg, fiber 0.9 g.

Each 250 ml serving contains 100 mg of soy isoflavones, and 8.75 g of soy proteins (35% RDA).

### Example 5

### Salad Dressing with Garlic Extract

The garlic flavored salad dressing of the invention is designed primarily for the diabetic and pre-diabetic populations and it includes no sugar, has a low fat content as to not contribute to the hypertriglyceridemia and/or hypercholesterolemia associated with diabetic conditions. Furthermore, the salad dressing is fortified with garlic extract, in order to further lower high blood cholesterol and LDL cholesterol levels as well as high blood pressure.

Ingredients: water, vinegar, olive oil, mustard, salt, wheat, soy beans, spices, fibers, plant stabilizers (E-405, guar gum), processed corn starch, citric acid, sucralose, beta-carotene, parsley, garlic extract.

Nutritional values (100 ml): Protein 0.84 g, Carbohydrates 3.6 g (of which 0.7 g dietary fibers), Fat 1.6 g (of which 12% saturated), Calcium 150 mg, Sodium 300 mg, and garlic extract 800 mg.

Each serving (15 mL) contains 800 mg of garlic extract, the average common level used in most clinical studies.

In comparison, a diet, low fat version of a salad dressing, marketed as contributing to weight loss and even as adequate to diabetics contain 20.9 g/100 g of fat, of which about 30% are saturated.

### Example 6

### Dairy Drink with Vitamins B6, B12 and Folic Acid

The fruit flavored milk drink of the invention is designed primarily for the diabetic and pre-diabetic populations and it includes no sugar, has a low fat content as to not contribute to the hypertriglyceridemia and/or hypercholesterolemia associated with diabetic conditions. Furthermore, the milk drink is fortified with vitamins B₆, B₁₂ and folic acid, in order to lower blood Homocysteine levels, an important CVD risk factor.

Ingredients: water, pasteurized milk, milk powder, polyalcohols, fruit flavors, fruit concentrate, natural food colorings, stabilizer E-410), Calcium (E-341), salt, vitamins B₆, B₁₂ and folic acid.

Nutritional values (100 ml): Protein 2.5 g, Carbohydrates 14.6 g, Fat 1.5 g (of which 15% saturated), Calcium 100 mg, Sodium 50 mg.

Each 250 ml serving contains B₆ (0.65 mg, 50% RDA), B₁₂ (1.2 mcg, 50% RDA), and folic acid (200 mg, 50% RDA).

### Example 7

### Barbecue Sauce with Lycopene

The Barbecue flavored sauce of the invention is designed primarily for the diabetic and pre-diabetic populations and it includes no sugar, has a low fat content as to not contribute to the hypertriglyceridemia and/or hypercholesterolemia associated with diabetic conditions. Furthermore, the sauce is fortified with lycopene, in order to lower oxidative damage, an important CVD risk factor.

Ingredients: water, tomato concentrate, maltitol, distilled vinegar, salt, mustard, tomato fibers, natural flavorings, spices, guar gum, paprika, pectin, lycopene.

Nutritional values (100 ml): Protein 1 g, Carbohydrates 36 g, Fat 0.4 g, Sodium 500 mg.

Each 15 ml serving contains 25 mg of lycopene (420 mg of 6% lycopene preparation).

### Example 8

### Light Whole Wheat Bread with Flax Seed Oil

The light whole wheat bread of the invention is designed primarily for the diabetic and pre-diabetic populations and it includes no sugar, has a low fat content as to not contribute to the hypertriglyceridemia and/or hypercholesterolemia associated with diabetic conditions. Furthermore, the bread's fat fraction is comprised of flax seed oil, instead of other vegetable oils and fats customarily used in bread recipes. Flax seed oil is highly rich in omega-3 linolenic acid, a precursor of the omega-3 LC-PUFAs, which lower blood triglycerides levels and reduce the risk of heart disease.

Ingredients: whole wheat flour, rye flour water, corn flour, rice flour, oat bran, yeast, barley malt, millet, wheat gluten, flax seeds, sunflower seeds, salt, soybean flour, emulsifier (E-472), acids (E-300, E-330), preservatives, flax seed oil.

Nutritional values (10 g): Protein 12.2 g, Carbohydrates 21.1 g, Fat 3.1 g, Sodium 410 mg, dietary fibers 13.8 g.

Each serving (slice) contains about 500 mg of flax seed oil, contributing about 250 mg of alpha-linolenic acid.

### Example 9

### Chocolate Flavored Biscuits with Selenium

The chocolate flavored biscuits of the invention are designed primarily for the diabetic and pre-diabetic populations and it includes no sugar, has a low fat content as to not contribute to the hypertriglyceridemia and/or hypercholesterolemia associated with diabetic conditions. Furthermore, the biscuits are fortified with selenium, an important mineral that plays an important role in many natural anti-oxidative enzymatic processes in order to lower oxidative damage, an important CVD risk factor.

Ingredients: Wheat flour, maltitol, vegetable oil, starch, cocoa powder, salt, lecithin, leavening agents (sodium bicarbonate, sodium pyrophosphate), acesulfam K, natural antioxidants (rosemary extract), sodium selenate.

Nutritional values (10 g): Protein 13.3 g, Carbohydrates 78 g, Fat 5.5 g (of which 20% saturated), Sodium 280 mg.

Each serving (4 biscuits, 20 g) contains 25 mcg of selenium (50% RDI).

In comparison, “regular” biscuits contain 12 g/100 g of fat, of which 35% are saturated fatty acids. Commercial diabetic biscuits, marketed as designed to address the needs of diabetics, contain log/100 g of fat, of which about 45% are saturated.

### Example 10

### Shortbread Cookies with PSE+DAG Combination

The shortbread cookies of the invention are designed primarily for the diabetic and pre-diabetic populations and it includes no sugar, has a low fat content as to not contribute to the hypertriglyceridemia and/or hypercholesterolemia associated with diabetic conditions. Furthermore, the fat fraction of the cookies is replaced by a combination of phytosterol esters (PSE) and diglycerides, characterized in higher PSE levels than DAG And clinically proven to lower both blood cholesterol and triglycerides levels as well as fight oxidative stress (see above).

Ingredients: Wheat flour, maltitol, sorbitol, polydextrose, maltodextrin, natural flavor, whey protein concentrate, salt, sodium bicarbonate, oat fiber, emulsifiers, aspartame, xanthan gum, acesulfame K, phytosterol esters of canola fatty acids, diglycerides and triglycerides of canola oil.

Nutritional values (10 g): Protein 7 g, Carbohydrates 80 g, Fat 6.6 g (of which 10% saturated), Sodium 250 mg.

Each serving (8 cookies, 30 g) contains 2 g of the PSE and DAG combination in canola oil, further contributing 1.3 g of phytosterol esters and 200-400 mg of DAG.

In comparison, commercial diabetic biscuits, marketed as designed to address the needs of diabetics, contain 22 g/100 g of fat, of which about 20% are saturated and of course do not contain any additional proactive ingredients to lower the risk of diabetic related CVD. Even “regular” shortbread cookies contain lower levels of fat than the commercial diabetic brand (17.2%) although the level of saturated fats is indeed higher (48% of total fat).

### Example 11

### Chocolate Covered Wafer with Vitamins E+C and Beta Carotene

The chocolate covered wafer of the invention is designed primarily for the diabetic and pre-diabetic populations and it includes no sugar, has a low fat content as to not contribute to the hypertriglyceridemia and/or hypercholesterolemia associated with diabetic conditions. Furthermore, the wafer is fortified by a combination of natural antioxidants, vitamin E, vitamin C, and beta-carotene, all promoting the lowering of CVD risks originating from oxidative damage.

Ingredients: maltitol, milk powder, cocoa butter, cocoa mass, vegetable oil, wheat flour, soya flour, nuts, emulsifiers, salt, flavors, leavening agents. Milk chocolate contained cocoa solids 30% min., vitamin E, vitamin C, beta-carotene.

Nutritional values (10 g): Protein 8 g, Polyols 39 g, Carbohydrates 15 g, Fat 15 g (of which 20% saturated), Sodium 71 mg.

Each serving (45 g wafer) contains 90 mg of vitamin C (100% RDI), 15 mg of vitamin E (100% RDA) and 16 mg of beta-carotene (100% RDI).

In comparison, commercial diabetic chocolate covered wafer, marketed as designed to address the needs of diabetics, contain 34 g/100 g of fat, of which 44% are saturated and of course do not contain any additional proactive ingredients to lower the risk of diabetic related CVD. A “regular” version of chocolate covered wafer contains slightly lower levels of fat (33 g/100 g) and slightly lower levels of saturated fat (“only” 42% of the total fat).

### Example 12

### Vanilla Flavored Sandwich Cookies with DHA/EPA

The vanilla flavored sandwich cookies of the invention are designed primarily for the diabetic and pre-diabetic populations and it includes no sugar, has a relatively low fat content as to not contribute to the hypertriglyceridemia and/or hypercholesterolemia associated with diabetic conditions. Furthermore, the cookies are fortified with omega-3 LC-PUFA, specifically DHA and EPA for promoting the lowering of CVD risk factors and also contributing to the maintenance or improvements of cognitive abilities.

Ingredients: maltitol, wheat flour, unsaturated vegetable oils, leavening agents, salt, lecithin, citric acid, flavors, Acesulfam K, antioxidant (rosemary extract), oil rich in DHA and EPA.

Nutritional values (10 g): Protein 6.7 g, Carbohydrates 70 g, Fat 12 g (of which 10% saturated), Sodium 157 mg.

Each serving (3 cookies, 33 g) contains 220 mg of DHA and EPA (33% RDA) originating from an omega-3 rich oil, either from cold water fish or from algal extracts.

In comparison, commercial diabetic sandwich cookies, marketed as designed to address the needs of diabetics, contain 20 g/100 g of fat, of which 25% are saturated and of course do not contain any additional proactive ingredients to lower the risk of diabetic related CVD or cognitive decline. A “regular” version of sandwich cookies contains only slightly higher levels of fat (22 g/100 g) and actually lower levels of saturated fat (18% of the total fat).

### Example 13

### Bitter Sweet Chocolate with Statins

The bitter sweet chocolate of the invention is designed primarily for the diabetic and pre-diabetic populations and includes no sugar, has a relatively low fat content as to not contribute to the hypertriglyceridemia and/or hypercholesterolemia associated with diabetic conditions. Furthermore, the chocolate is fortified with statins for promoting the lowering of total and LDL cholesterol, which are important CVD risk factors.

Ingredients: maltitol, cocoa mass, cocoa butter, emulsifiers, flavors, statins.

Nutritional values (10 g): Protein 5 g, Carbohydrates 61 g, Fat 25 g (of which 40% saturated), Sodium 8 mg.

Each serving (20 g) contains a low dosage of statins, such as Pravastatin or Lovastatin, for example 2.5 mg/serving, to maintain a lowering of cholesterol effect. Higher doses of the statins can be delivered in said food product in case the number or variety of diabetics' foods delivering such active ingredients is very low and/or limited.

In comparison, commercial diabetic bitter sweet chocolate, marketed as designed to address the needs of diabetics, contain 31 g/100 g of fat, of which 61% are saturated and of course do not contain any additional proactive ingredients to lower the risk of diabetic related CVD. A “regular” version of bittersweet chocolate contains actually slightly lower levels of fat (28 g/100 g) and similar levels of saturated fat (60% of the total fat).

### Example 14

### Chocolate Spread with Canola Oil and Polyphenols

The chocolate spread of the invention is designed primarily for the diabetic and pre-diabetic populations and includes no sugar, has a relatively low fat content as to not contribute to the hypertriglyceridemia and/or hypercholesterolemia associated with diabetic conditions. Furthermore, the chocolate spread's fat fraction utilizes primarily canola oil, characterized in high levels of unsaturated fatty acids and is further fortified with polyphenols characterized in strong antioxidation activity for fighting CVD risk factors. As seen in Example 1, the mere utilization of unsaturated fats and oils (HMUFA in this case) as the major fat of the diet is not sufficient to promote a significant improvement in CVD risk factors, such as total cholesterol, non-HDL cholesterol, and total cholesterol/HDL ratio, and hence additional proactive agents are necessary in order to exhibit lowering of the risk.

Ingredients: maltitol, vegetable oils and fats (mainly canola oil), hazelnuts, soy flour, insulin (dietary fiber), cocoa powder, soy lecithin (emulsifier), flavors, polyphenols antioxidants (grape extract).

Nutritional values (100 g): Protein 6 g, Carbohydrates 49 g, Fat 20 g (of which 10% saturated), Sodium 1 mg.

Each serving (25 g) contains 70 mg of polyphenols antioxidants such as grape extract, pomegranate extract, olive hydroxytyrosol, etc.

In comparison, commercial diabetic chocolate spread, marketed as designed to address the needs of diabetics, contain 38 g/100 g of fat, and of course do not contain any additional proactive ingredients to lower the risk of diabetic related CVD. The “regular” version of a chocolate spread contains actually lower levels of fat (25.5 g/100 g).

### Example 15

### Chocolate Flavored Wafers with Soy Proteins and Phytosterols

The chocolate flavored wafers of the invention are designed primarily for the diabetic and pre-diabetic populations and include no sugar, has a relatively low fat content as to not contribute to the hypertriglyceridemia and/or hypercholesterolemia associated with diabetic conditions. Furthermore, the wafers are fortified with soy proteins and wood phytosterols, synergistically contributing to balanced blood lipid profiles and reduce the risk of CVD.

Ingredients: maltitol, vegetable oils and fats, wheat flour, soy flour, cocoa powder, emulsifier (lecithin), flavors, salt, leavening agents, baking improver (protease), soy proteins isolate, wood phytosterols.

Nutritional values (10 g): Protein 6 g, Carbohydrates 66 g, Fat 15 g (of which 13% saturated), Sodium 60 mg.

Each serving (4 wafers) contains 100 mg of wood phytosterols (12.5% RDA) and 2.5 g of soy proteins (10% RDA) These levels of active ingredients are possible when a variety of food products supply these ingredients through the daily nutrition.

In comparison, commercial diabetic chocolate flavored wafers, marketed as designed to address the needs of diabetics, contain 36 g/100 g of fat, of which 82% (!!!) are saturated and of course do not contain any additional proactive ingredients to lower the risk of diabetic related CVD. A “regular” version of chocolate flavored wafers actually contains lower levels of fat (18.4 g/100 g) and actually lower levels of saturated fat (28% of the total fat).

### Example 16

### Canola Oil Low Fat Vanilla Chocolate Ice Cream and Soy Proteins

The low fat vanilla chocolate-ice cream of the invention is designed primarily for the diabetic and pre-diabetic populations and include no sugar, has a low fat content as to not contribute to the hypertriglyceridemia and/or hypercholesterolemia associated with diabetic conditions. Furthermore, the ice cream's fat fraction is mainly composed of an unsaturated vegetable oil, such as canola oil. The ice cream is further fortified with soy proteins, the latter contributing to balanced blood lipid profiles and reduces the risk of CVD.

Ingredients: milk, milk solids, polydextrose, maltitol, insulin (dietary fiber), cocoa powder, glycerol, vegetable oils, emulsifiers, stabilizers, flavors, sweeteners (Acesulfam K, sucralose), soy proteins.

Nutritional values (100 g): Protein 10 g, Carbohydrates 12.8 g, Fat 1.5 g (of which 20% saturated), Fibers 5 g, Sodium 70 mg, Calcium 114 mg.

Each serving (150 g) contains 13.2 g of soy proteins (50% RDA).

In comparison, commercial diabetic ice cream, marketed as designed to address the needs of diabetics, contain only 1.5 g/100 g of fat, of which 74% (!!!) are saturated and of course do not contain any additional proactive ingredients to lower the risk of diabetic related CVD.

### Example 17

### Yoghurt Drink with Phosphatidylserine

The fruit flavored yoghurt drink of the invention is designed primarily for the diabetic and pre-diabetic populations and it includes no sugar, has a low fat content as to not contribute to the hypertriglyceridemia and/or hypercholesterolemia associated with diabetic conditions. Furthermore, the yoghurt drink is fortified with phosphatidylserine, providing 100% of its minimal recommended daily dose, in order to further protect against cognitive decline or to address different stages of cognitive decline.

Ingredients: pasteurized milk, maltitol, fruit puree/concentrate, milk powder, processed starches (E1422, E1442), natural stabilizer (E440), flavor agents, citric acid, phosphatidylserine.

Nutritional values (100 ml): Protein 3.1 g, Carbohydrates 16.4 g, Fat 1.5 g (1.18 g milk fat, 0.32 g soy phytosterols), Calcium 110 mg, Sodium 76 mg.

Each 250 ml serving contains 100 mg of soybean derived phosphatidylserine (100% of the minimal recommended daily dose).

### Example 18

### A Functional Chocolate Bar for Diabetics

The bar is a chocolate flavor functional bar, which provides 50% of the daily-recommended dose of phytosterol-esters. The bar includes a combination of phytosterol esters and DAG dissolved in canola oil (Preparation A). Said combination includes 70% wt of phytosterol esters and about 8% wt of DAG with a total fatty acid profile of Canola oil.

Typical composition of bar: Combination of phytosterol esters and DAG (about 1 g), (Preparation A). Sugar Alcohols (Maltitol, Maltitol Syrup), Soy Protein Isolate, Dietary fibers (nulin, Polydextrose), Canola oil, Cocoa Powder, Cocoa Butter, Cocoa mass, Sweeteners (Sucralose), Emulsifiers, Flavourings. The total weight of each bar is 50 g.

Active ingredients (g/serving): Plant sterol esters (0.65 g/serving), Diglycerides (0.1 g/serving).

### Nutritional Information:

| | |
|---|---|
| per 100 g | per bar |

| | | | | |
|---|---|---|---|---|
| Energy | 260 | Kcal | 130 | kcal |
| Protein | 16 | g | 8 | g |
| Carbohydrate | 46 | g | 23 | g |
| Total Fat | 2.8 | g | 1.4 | g |
| Saturated | 1 | g | 0.5 | g |
| Fat | | | | |

### Vitamins and Minerals:

| | Net per 1 unit of | % from RDA |
|---|---|---|
| Vitamin/Mineral | product [mg] | In 1 unit of product |
| A | 0.16 | 20 |
| B1 | 0.28 | 20 |
| B2 | 0.32 | 20 |
| B3 | 3.60 | 20 |
| B6 | 0.40 | 20 |
| B12 | 0.0002 | 20 |
| Biotin | 0.03 | 20 |
| C | 12.00 | 20 |
| Ca-pantothenate | 1.32 | 20 |
| E | 2.00 | 20 |
| Folic acid | 0.04 | 20 |
| Ca | 160.00 | 20 |
| Mg | 18.00 | 6 |
| Fe | 2.80 | 20 |
| Zn | 3.00 | 20 |
| Cu | 0.08 | 4 |
| P | 160.00 | 20 |
| I | 0.03 | 20 |
| Mo | 0.04 | 2 |

1. A food product characterized by:
having a low glucose content of 0 to 5% glucose, preferably 0 to 0.5% glucose, more preferably being glucose free;
having a balanced fat content of less than 10%; and
comprising at least one dietary or pharmaceutical substance which is proactive towards diabetes and any of its complications, and/or towards a condition leading to diabetes;
wherein said food product addresses health needs of diabetics and/or prevents onset of diabetes in healthy individuals or individuals prone to diabetes.

2. The food product of claim 1, being glucose free.

3. A food product characterized by:
being sugar free;
having a balanced fat content of less than 10%; and
comprising at least one dietary or pharmaceutical substance which is proactive towards
diabetes and any of its complications, and/or towards a condition leading to diabetes;
wherein said food product addresses health needs of diabetics and/or prevents onset of diabetes in healthy individuals or individuals prone to diabetes.

4. The food product of claim 1, wherein said fat content comprises saturated fats at less than 25%, preferably below 10% of the total fats.

5. The food product of claim 1, wherein said diabetes-proactive dietary or pharmaceutical substance is any one of a naturally occurring lipid, a synthetic or mimetic lipid which is not digestible by humans and interferes with body weight gain/loss, antioxidants, and pharmaceuticals, and any mixture thereof.

6. The food product of claim 5, wherein:
said lipid is any one of diacylglycerols, particularly 1,3-diacylglycerols, phytosterols and phytosterol esters, phytostanols and phytostanol esters, polycosanol, omega-3 fatty acids and their derivatives, particularly long-chain polyunsaturated fatty acids (LC-PUFA), polyunsaturated fatty acids (PUFA), particularly alpha-linolenic acid, and conjugated linolenic acid (CLA); and/or
said non-digestible synthetic lipid or lipid mimetic is an alpha branched triglyceride or olestra, respectively; and/or
said antioxidants is any one of rosemary extract, lycopene, zeaxanthin, selenium, zinc, vitamin C, vitamin E and other tocopherols, coenzyme Q₁₀, beta-carotene, polyphenols, particularly hydroxytyrosol; and/or
said pharmaceutical is any one of statins, ezetimibe, a drug controlling lipids profile or other biomarker related to cardiovascular diseases; and/or
any mixtures thereof, wherein said substance may be optionally dispersed or dissolved in an edible oil or fat.

7. The food product of claim 1, wherein said substance is a mixture of phytosterol and/or phytostanol esters with 1,3-diacylglycerides, optionally dispersed or dissolved in an edible oil or fat.

8. The food product of claim 6, wherein said mixture is dispersed or dissolved in an edible oil or fat.

9. The food product of claim 7, further comprising at least one pharmaceutical drug.

10. The food product of claim 1, which is functional in the treatment and/or prevention of cardiovascular disease (CVD) and/or its risk factors hyperlipidemia, dyslipidemia, oxidative stress and atherosclerosis, particularly in diabetic or diabetes-prone individuals.

11. The food product of claim 1, which is functional in the treatment and/or prevention of hyperlipidemia, dyslipidemia, oxidative stress and atherosclerosis, particularly in diabetic or diabetes-prone individuals.

12. The food product of claim 1, which is functional in at least one of the following, reducing the total cholesterol serum level, reducing the non-HDL cholesterol serum level, reducing the total cholesterol/HDL ratio and reducing triglycerides serum level in an overweight, and/or obese, and/or diabetic, and/or diabetic-prone subject.

13. The food product of claim 1, which is functional in reducing the body weight, and/or inhibiting body weight gain, and/or reducing insulin resistance in an obese and/or in a subject with metabolic imbalances such as diabetes and/or an individual prone to diabetes or obesity.

14. The food product of claim 1, which is functional in remodeling body fat distribution, suppressing WAT accumulation and reducing visceral fat accumulation in an obese and/or overweight subject and/or diabetic subject and/or pre-diabetic subject and/or an individual prone to diabetes or obesity.

15. The food product of claim 1, which is functional in reducing the risk of life threatening long term diabetes complications and deterioration of quality of life in an obese and/or diabetic subject.

16. The food product of claim 15, wherein said complication is from macrovascular, microvascular or neurological origin.

17. The food product of claim 16, wherein said complication is selected From the group of retinopathy, nephropathy, diseases of the large vessels supplying the legs (lower extremity arterial disease), coronary heart or artery diseases, cerebrovascular diseases and disturbed neural function afflictions, decline of cognitive functions, or any other condition which may lead to blindness, end-stage kidney disease (ESRD) and amputations, myocardial infractions and stroke.

18. The food product of claim 1, which is functional in ameliorating hyperinsulinemia in an insulin-resistant subject.

19. The food product of claim 1, which is functional in ameliorating hyperinsulinemia or preventing progression of insulin resistance in an obese subject prone to develop diabetes.

20. The food product of claim 1, further comprising active agents which are functional in the prevention and/or treatment of acute cognitive decline.

21. The food product of claim 20, wherein said active agents are phosphatidylserine, ginko biloba, brahmi (Bacopa monnieri) or omega-3 containing fiats.

22. The food product of claim 1, further comprising active agents which are functional in the prevention and/or treatment of acute ophthalmic conditions associated with diabetes Type 2, wherein said active agents are vitamins, antioxidants, and other natural eye-health promoting ingredients or extracts.

23. The food product of claim 1, wherein said food product is any one of dairy products, bakery products, condiments, beverages and drinks, snacks, candies, ice-creams and frozen desserts, morning cereals, nutrition bars, snack bars chocolate products, prepared foods, grain products and pasta, soups, sauces and dressings, confectionery products, oils and fats products, dairy and milk drinks, soy milk and soy dairy-like products, frozen food products, prepared meals and meal replacements, meat products, cheeses, yoghurts, breads and rolls, yeast products, cakes and cookies and crackers.

24. Use of a food product of claim 1 in the nutrition of diabetics and/or for preventing onset of diabetes in healthy individuals or individuals prone to diabetes.

25. The use of a food product of claim 1, in the treatment and/or prevention of cardiovascular disease (CVD) and/or its risk factors hyperlipidemia, dyslipidemia, oxidative stress and atherosclerosis, particularly in diabetic or diabetes-prone individuals.

26. The use of a food product of claim 1, in the treatment and/or prevention of hyperlipidemia, dyslipidemia, oxidative stress and atherosclerosis, particularly in diabetic or diabetes-prone individuals.

27. The food product of claim 1, which is functional in at least one of the following: reducing the total cholesterol serum level, reducing the non-HDL cholesterol serum level, reducing the total cholesterol/HDL ratio and reducing triglycerides serum level in an overweight, and/or obese, and/or diabetic, and/or diabetic-prone subject.

28. The use of a food product of claim 1, for reducing the body weight, and/or inhibiting body weight gain, and/or reducing insulin resistance in an obese and/or in a subject with metabolic imbalances such as diabetes and/or an individual prone to diabetes or obesity.

29. The use of a food product of claim 1, in remodeling body fat distribution, suppressing WAT accumulation and reducing visceral fat accumulation in an obese and/or overweight subject and/or diabetic subject and/or pre-diabetic subject and/or an individual prone to diabetes or obesity.

30. The use of a food product of claim 1, in reducing the risk of life threatening long term diabetes complications and deterioration of quality of life in an obese and/or diabetic subject.

31. The use of claim 30, wherein said complication is from macrovascular, microvascular or neurological origin.

32. The use of claim 30, wherein said complication is selected from the group of retinopathy, nephropathy, diseases of the large vessels supplying the legs (lower extremity arterial disease), coronary heart or artery diseases, cerebrovascular diseases and disturbed neural function afflictions, decline of cognitive functions, or any other condition which may lead to blindness, end-stage kidney disease (ESRD) and amputations, myocardial infractions and stroke.

33. The use of claim 1, which is functional in ameliorating hyperinsulinemia in an insulin-resistant subject.

34. The use of claim 1, which is functional in ameliorating hyperinsulinemia or preventing progression of insulin resistance in an obese subject prone to develop diabetes.

35. The use of claim 1, wherein said food product further comprises active agents which are functional in the prevention and/or treatment of acute cognitive decline.

36. The use of claim 35, wherein said active agents are phosphatidylserine, grinko biloba, brahmi (Bacopa monnieri) or omega-3 containing fats.

37. The use of claim 24, wherein said food product further comprises active agents which are functional in the prevention and/or treatment of acute ophthalmic conditions associated with diabetes Type 2, wherein said active agents are vitamins, antioxidants, and other natural eye-health promoting ingredients or extracts.

38. The use of claim 24, wherein said food product is any one of dairy products, bakery products, condiments, beverages and drinks, snacks, candies, ice-creams and frozen desserts, morning cereals, nutrition bars, snack bars chocolate products, prepared foods, grain products and pasta, soups, sauces and dressings, confectionery products, oils and fats products, dairy and milk drinks, soy milk and soy dairy-like products, frozen food products, prepared meals and meal replacements, meat products, cheeses, yoghurts, breads and rolls, yeast products, cakes and cookies and crackers.
